(19) United States
(12) Patent Application Publication
(10) Pub. No.: US 2024/0344029 A1
(43) Pub. Date: Oct. 17, 2024

(54) STEM CELL DIFFERENTIATION BY CONTROLLING NUCLEAR CURVATURE

(71) Applicant: THE ROYAL INSTITUTION FOR THE ADVANCEMENT OF LEARNING / MCGILL UNIVERSITY, Montréal (CA)

(72) Inventors: Allen EHRLICHER, Montreal (CA); Ajinkya GHAGRE, Montreal (CA)

(21) Appl. No.: 18/632,921

(22) Filed: Apr. 11, 2024

Related U.S. Application Data
(60) Provisional application No. 63/496,148, filed on Apr. 14, 2023.

(51) Int. Cl. C12N 5/077; C12N 5/0793

(52) U.S. Cl. CPC C12N 5/0654; C12N 5/0619; C12N 5/0653; C12N 2500/30; C12N 2500/38; C12N 2500/42; C12N 2506/1346; C12N 2527/00; C12N 2531/00

(57) ABSTRACT

There is provided a method of selectively differentiating mesenchymal stem cells into a first predetermined cell lineage associated with the nuclear localization of Yes-associated protein (YAP) or into a second predetermined cell lineage associated with the cytoplasmic localization of YAP. The curvature of the nucleus is controlled to have a maximum nuclear curvature (Kmax) of at least 0.5 μm⁻¹ to select for the first predetermined cell lineage, or a Kmax that does not exceed 0.5 μm⁻¹ to select for the second predetermined cell lineage. The mesenchymal stem cells having a controlled nuclear curvature are incubated in a media with or without differentiation additives to obtain the first predetermined cell lineage or the second predetermined cell lineage.

### CROSS REFERENCE TO A RELATED APPLICATION

This disclosure claims priority from U.S. provisional application No. 63/496,148 filed Apr. 14, 2023, which is incorporated herein by reference in its entirety.

### TECHNICAL FIELD

This disclosure relates to the field of stem cell differentiation, particularly differentiations associated with the localization of Yes-associated protein (YAP).

### BACKGROUND OF THE ART

Mesenchymal stem cells (MSC) have the ability to differentiate into multiple cell lineages. They can be isolated from multiple different sources and can be cultured in vitro with ease. These features have made MSCs one of the main cell lines for tissue engineering and regenerative medicine applications. The controlled differentiation of MSCs into their target cell populations requires the identification of specific molecules or cues to regulate differentiation. Particularly, diverse mechanical cues such as matrix stiffness, viscosity, topography, surface curvature, cell spread area, cell volume, geometric shape, compression, and tension can affect the differentiation of MSCs. Mechanically directed differentiation requires contractility generated by actomyosin interactions to sense the mechanical cues and determine its differentiation fate. For example, MSCs cultured on a stiff matrix differentiate into osteocytes, in contrast, MSCs cultured on a softer matrix differentiate into adipocytes. Nevertheless, contractile inhibition abrogates any matrix stiffness sensitivity causing MSCs differentiate into adipocytes irrespective of matrix stiffness. This indicates that matrix stiffness is an indirect cellular cue in differentiation, and does not stipulate lineage but rather biases cell activity. It would be desirable to determine how contractility translates into the genetic expression of differentiation markers in MSCs to improve the consistency and efficiency of MSC differentiation.

### SUMMARY

There is provided a method of selectively differentiating mesenchymal stem cells into a first predetermined cell lineage associated with the nuclear localization of Yes-associated protein (YAP) or into a second predetermined cell lineage associated with the cytoplasmic localization of YAP. The curvature of the nucleus is controlled to have a maximum nuclear curvature (Kmax) of at least 0.5 μm⁻¹ to select for the first predetermined cell lineage, or a Kmax that does not exceed 0.5 μm⁻¹ to select for the second predetermined cell lineage. The mesenchymal stem cells having a controlled nuclear curvature are incubated in a media with or without differentiation additives to obtain the first predetermined cell lineage or the second predetermined cell lineage. The first predetermined cell lineage can be an osteocyte lineage and the second predetermined cell lineage can an adipocyte lineage or a neuronal lineage. In some embodiments, the Kmax to select for the first predetermined cell lineage is at least 0.5 μm⁻¹, preferably at least 0.7 μm⁻¹. Example of supplements for the incubation include β-glycerol phosphate, ascorbic acid, and dexamethasone.

In some embodiments, the controlling of the curvature comprises physically confining the mesenchymal stem cells, for example on micro-patterns. Preferably, the micro-patterns control cell adhesion promoting the cell's ability to compress the nucleus and increasing YAP activity to select for the first predetermined cell lineage, for example by compressing the nucleus of the mesenchymal stem cells. The micro-patterns can control cell adhesion and can restrict the cell's ability to compress the nucleus and reducing YAP activity to select for the second predetermined cell lineage. This can be done by confining the mesenchymal stem cells inside microchannels. In some embodiments, the controlling of the curvature comprises applying a pressure on the nucleus of the mesenchymal stem cells. The pressure can be applied vertically (with physical means for example), laterally (for example using fibers that are preferably electrospun, polymerized or extruded), or the pressure can be an osmotic pressure (for example applied using polyethylene glycol (PEG)). In some embodiments, the volume of the nucleus is controlled to be 50% less than the physiological volume of the nucleus to select for the first predetermined cell lineage.

As used herein the term “selectively” when used in the context of selecting a differentiated lineage is defined as having the resulting cultured population of cells containing at least 80%, preferably at least 90%, more preferably at least 95% of the cells being of the selected lineage after differentiation.

Many further features and combinations thereof concerning the present improvements will appear to those skilled in the art following a reading of the instant disclosure.

### DESCRIPTION OF THE DRAWINGS

FIG. 1A is a fluorescence microscopy image of a MSC transfected (highly contractile cell) with iRFP YAP and stained with Hoechst on 2 kPa traction force microscopy (TFM) substrates.

FIG. 1B is a 3D Nuclear volumes image reconstructed from XYZ confocal stacks, showing a flat nucleus with small nuclear volume for the cell of FIG. 1A.

FIG. 1C is a traction stress map and strain energy obtained from traction force microscopy (TFM) shows high traction stress and strain energy applied by the cell of FIG. 1A.

FIG. 1D is a fluorescence microscopy image of a MSC transfected (low contractility cell) with iRFP YAP and stained with Hoechst on 2 kPa traction force microscopy (TFM) substrates.

FIG. 1E is a 3D Nuclear volumes image reconstructed from XYZ confocal stacks, showing a flat nucleus with small nuclear volume for the cell of FIG. 1D.

FIG. 1F is a traction stress map and strain energy obtained from traction force microscopy (TFM) shows high traction stress and strain energy applied by the cell of FIG. 1D.

FIG. 1G is a graph showing an increase in cell strain energy decreases nuclear volumes (Wildtype MSCs, n=73, R²=coefficient of determination, R₀=Rate Constant, p=Pearson's correlation coefficient).

FIG. 1H is a graph showing a decrease in nuclear volumes increases YAP Ratio (Wildtype MSCs, n=73, R²=coefficient of determination, R₀=Rate Constant, p=Pearson's correlation coefficient).

FIG. 2A is a microscopy image showing a Hoechst-stained nucleus of MSC with high contractility in XY, YZ and XZ projection.

FIG. 2B is a microscopy image showing nuclear curvatures for the Hoechst-stained nucleus of FIG. 2A in XY, YZ and XZ projection.

FIG. 2C is a microscopy image showing a Hoechst-stained nucleus of MSC with low contractility in XY, YZ and XZ projection.

FIG. 2D is a microscopy image showing nuclear curvatures for the Hoechst-stained nucleus of FIG. 2C in XY, YZ and XZ projection.

FIG. 2E is a graph showing an increase in cell strain energy increases Kmax XZ. (Wildtype MSCs, n=73, R²=coefficient of determination, p=Pearson's correlation coefficient).

FIG. 2F is a graph showing an increase in Kmax XZ increases YAP Ratio (Wildtype MSCs, n=73, R²=coefficient of determination, p=Pearson's correlation coefficient).

FIG. 3A is a fluorescence microscopy image of MSCs transfected with iRFP YAP and stained with Hoechst under hyperosmotic pressure in suspension.

FIG. 3B is a 3D Nuclear volumes image reconstructed from XYZ confocal stacks for the cell of FIG. 3A.

FIG. 3C is a microscopy image showing a Hoechst-stained nucleus of FIG. 3A in XY, YZ and XZ projection.

FIG. 3D is a microscopy image showing nuclear curvatures for the Hoechst-stained nucleus of FIG. 3C in XY, YZ and XZ projection.

FIG. 3E is a fluorescence microscopy image of MSCs transfected with iRFP YAP and stained with Hoechst under hyperosmotic pressure in suspension with 10% polyethylene glycol (PEG).

FIG. 3F is a 3D Nuclear volumes image reconstructed from XYZ confocal stacks for the cell of FIG. 3E.

FIG. 3G is a microscopy image showing a Hoechst-stained nucleus of FIG. 3F in XY, YZ and XZ projection.

FIG. 3H is a microscopy image showing nuclear curvatures for the Hoechst-stained nucleus of FIG. 3G in XY, YZ and XZ projection.

FIG. 3I is a fluorescence microscopy image of MSCs transfected with iRFP YAP and stained with Hoechst under hyperosmotic pressure as an adherent culture.

FIG. 3J is a 3D Nuclear volumes image reconstructed from XYZ confocal stacks for the cell of FIG. 3I.

FIG. 3K is a microscopy image showing a Hoechst-stained nucleus of FIG. 3I in XY, YZ and XZ projection.

FIG. 3L is a microscopy image showing nuclear curvatures for the Hoechst-stained nucleus of FIG. 3K in XY, YZ and XZ projection.

FIG. 3M is a fluorescence microscopy image of MSCs transfected with iRFP YAP and stained with Hoechst under hyperosmotic pressure as an adherent culture with 10% polyethylene glycol (PEG).

FIG. 3N is a 3D Nuclear volumes image reconstructed from XYZ confocal stacks for the cell of FIG. 3M.

FIG. 3O is a microscopy image showing a Hoechst-stained nucleus of FIG. 3N in XY, YZ and XZ projection.

FIG. 3P is a microscopy image showing nuclear curvatures for the Hoechst-stained nucleus of FIG. 3O in XY, YZ and XZ projection.

FIG. 3Q is a graph showing that for suspended MSCs, no change in Kmax XZ with decreasing nuclear volumes after PEG.

FIG. 3R is graph showing that for adherent MSCs, Kmax XZ increases with decreasing nuclear volumes after PEG.

FIG. 3S is a graph showing an increase in Kmax XZ increases percent change in YAP Ratio. (Adherent cells n=45 above solid line, Suspended cells n=40 below solid line, R²=coefficient of determination, p=Pearson's correlation coefficient).

FIG. 3T is a graph showing adherent cells exhibit anisotropic nuclear deformation with highest percent deformation in Z, whereas suspended cells exhibit isotropic nuclear deformation with equal percent nuclear deformation in all the planes.

FIG. 4A is a microscopy image showing MSCs transfected with iRFP YAP and stained with Hoechst confined on fibronectin line patterns with decreasing thickness (1.5, 2, 4, 6 and 8 μm line width).

FIG. 4B is a microscopy image showing a Hoechst-stained nucleus of the MSC confined in 1.5 μm line width of FIG. 4A in XY, YZ and XZ projection.

FIG. 4C is a microscopy image showing nuclear curvatures for the Hoechst-stained nucleus of FIG. 4B in XY, YZ and XZ projection.

FIG. 4D is a microscopy image showing a Hoechst-stained nucleus of the MSC confined in 2 μm line width of FIG. 4A in XY, YZ and XZ projection.

FIG. 4E is a microscopy image showing nuclear curvatures for the Hoechst-stained nucleus of FIG. 4D in XY, YZ and XZ projection.

FIG. 4F is a microscopy image showing a Hoechst-stained nucleus of the MSC confined in 4 μm line width of FIG. 4A in XY, YZ and XZ projection.

FIG. 4G is a microscopy image showing nuclear curvatures for the Hoechst-stained nucleus of FIG. 4F in XY, YZ and XZ projection.

FIG. 4H is a microscopy image showing a Hoechst-stained nucleus of the MSC confined in 6 μm line width of FIG. 4A in XY, YZ and XZ projection.

FIG. 4I is a microscopy image showing nuclear curvatures for the Hoechst-stained nucleus of FIG. 4H in XY, YZ and XZ projection.

FIG. 4J is a microscopy image showing a Hoechst-stained nucleus of the MSC confined in 8 μm line width of FIG. 4A in XY, YZ and XZ projection.

FIG. 4K is a microscopy image showing nuclear curvatures for the Hoechst-stained nucleus of FIG. 4J in XY, YZ and XZ projection.

FIG. 4L is a graph showing increasing Kmax XY increases YAP Ratio in MSCs confined on fibronectin line patterns with decreasing thickness (n=55, R²=coefficient of determination, R₀=Rate Constant, p=Pearson's correlation coefficient).

FIG. 4M is a graph showing the YAP ratio in function of the nuclear volume.

FIG. 4N is a graph showing the YAP ratio in function of the Kmax XY.

FIG. 5A is a microscopy image of MSCs adhered to patterned fibronectin lines of controlled thickness show increased YAP nuclear localization on thinnest pattern of 1.5 μm thickness.

FIG. 5B is a microscopy image showing XY nuclear curvatures of MSCs adhered to patterned fibronectin lines, show increase in nuclear curvature on thinnest pattern of 1.5 μm thickness

FIG. 5C is a Forster Resonance Energy Transfer (FRET) image of MSCs on 8 μm and 1.5 μm thick fibronectin lines.

FIG. 5D is a graph showing decreasing FRET mean intensity increases YAP Ratio in MSCs confined on fibronectin line patterns with decreasing thickness (n=44, R²-coefficient of determination, p=Pearson's correlation coefficient).

FIG. 5E is a graph showing increasing Kmax XY decreases FRET mean intensity in MSCs confined on fibronectin line patterns with decreasing thickness (n=44, R²-coefficient of determination, p=Pearson's correlation coefficient).

FIG. 5F is a graph showing increasing Kmax XY increases YAP Ratio in MSCs confined on fibronectin line patterns with decreasing thickness (n=44, R²=coefficient of determination, p=Pearson's correlation coefficient).

FIG. 5G is a microscopy image showing MSCs transfected with Nesprin Headless Control confined on thin and thick line patterns (1.5 and 8 μm) which show similar low nuclear membrane tension on both thin and thick line patterns.

FIG. 5H is a graph showing the FRET mean intensity increases with increasing pattern width from 1.5-8 μm for MSCs with Nesprin TS.

FIG. 6A is a microscopy image of a MSC differentiated on a thickness line micropatterns conferring the MSC a Kmax XY of 0.25 μm⁻¹ for two weeks and stained with adipogenic (Lpdx).

FIG. 6B is a microscopy image of the MSC of FIG. 6A stained with osteogenic (ALP) differentiation markers.

FIG. 6C is a microscopy image of the MSC of FIG. 6A stained with Hoechst for nuclei.

FIG. 6D is a microscopy image of a MSC differentiated on a thickness line micropatterns conferring the MSC a Kmax XY of 0.4 μm⁻¹ for two weeks and stained with adipogenic (Lpdx).

FIG. 6E is a microscopy image of the MSC of FIG. 6D stained with osteogenic (ALP) differentiation markers.

FIG. 6F is a microscopy image of the MSC of FIG. 6D stained with Hoechst for nuclei.

FIG. 6G is a microscopy image of a MSC differentiated on a thickness line micropatterns conferring the MSC a Kmax XY of 0.54 μm⁻¹ for two weeks and stained with adipogenic (Lpdx).

FIG. 6H is a microscopy image of the MSC of FIG. 6G stained with osteogenic (ALP) differentiation markers.

FIG. 6I is a microscopy image of the MSC of FIG. 6G stained with Hoechst for nuclei.

FIG. 6J is a microscopy image of a MSC differentiated on a thickness line micropatterns conferring the MSC a Kmax XY of 0.7 μm⁻¹ for two weeks and stained with adipogenic (Lpdx).

FIG. 6K is a microscopy image of the MSC of FIG. 6J stained with osteogenic (ALP) differentiation markers.

FIG. 6L is a microscopy image of the MSC of FIG. 6J stained with Hoechst for nuclei.

FIG. 6M is a graph showing that for differentiated MSCs, ALP/Lpdx Ratio increases with increasing nuclear curvature in XZ plane. The differentiated cell images in FIGS. 6A, 6D, 6G and 6J are respectively identified as A, B, C, and D on the graph (n=44, R₀=Rate Constant).

FIG. 7A is a microscopy image showing MSCs that were plastic-adherent when maintained in standard culture conditions.

FIG. 7B is a fluorescent microscopy image showing that MSCs expressed CD44 and lacked expression of CD45 surface marker.

FIG. 7C is a microscopy image showing cells differentiated into osteocytes.

FIG. 7D is a microscopy image showing cells differentiated into adipocytes.

FIG. 8 is a graph showing that dynamic vertical compression reveals a time lag between nuclear compression and YAP translocation. Time-course of Nuclear Curvature and YAP Ratio under hyperosmotic conditions with 10% PEG400 (1.62 MPa osmotic pressure, duration: ˜900 secs, time interval: ˜ 0.1 secs).

FIG. 9A shows the XYZ stacks showing XY image slices over complete Z stack.

FIG. 9B is a schematic showing the calculation in XY of maximum and minimum nuclear curvatures (Kmin and Kmax respectively).

FIG. 9C is a schematic showing the addition of the Z stacks on the schematic of FIG. 9B to obtain a three dimensional visualization.

FIG. 9D is a graph showing the nuclear curvature distribution of wildtype MSCs (contractility present) and CytoD (contractility inhibited) treated MSCs measured in XY slices from XYZ stacks, cells with different contractility show similar distribution of curvature when quantified from XY slices.

FIG. 9E shows the XYZ stacks showing XZ image slices over complete Y stack.

FIG. 9F is a schematic showing the calculation in XZ of Kmin and Kmax.

FIG. 9G is a schematic showing the addition of the Y stacks on the schematic of FIG. 9F to obtain a three dimensional visualization.

FIG. 9H is a graph showing the nuclear curvature distribution of wildtype MSCs (contractility present) and CytoD (contractility inhibited) treated MSCs measured in XZ slices from XYZ stacks, cells in the presence of contractility show a separate distribution of high curvature values when quantified from XZ slices.

FIG. 9I shows the XYZ stacks showing YZ image slices over complete Y stack.

FIG. 9J is a schematic showing the calculation in YZ of Kmin and Kmax.

FIG. 9K is a schematic showing the addition of the X stacks on the schematic of FIG. 9J to obtain a three dimensional visualization.

FIG. 9L is a graph showing the nuclear curvature distribution of wildtype MSCs (contractility present) and CytoD (contractility inhibited) treated MSCs measured in YZ slices from XYZ stacks, cells in the presence of contractility show a separate distribution of high curvature values when quantified from YZ slices.

FIG. 10A is a graph showing that the YAP ratio increases with increasing nuclear flatness.

FIG. 10B is a graph showing that the YAP ratio increases with decreasing nuclear height.

FIG. 10C is a graph showing that no significant relation between nuclear surface area and the YAP ratio was found.

FIG. 10D is a graph showing that the YAP Ratio increases with increasing nuclear eccentricity.

FIG. 10E is a graph showing that no significant relation between the nuclear aspect ratio and the YAP ratio was observed.

FIG. 11A is a microscopy MSCs stained with anti-YAP antibody and hoechst on fibronectin-treated glass coverslips, showing high nuclear YAP hence a high YAP Ratio.

FIG. 11B is a microscopy image showing a 3D Nuclear volumes reconstructed from confocal stacks show flatter nuclei with low nuclear volume.

FIG. 11C is a MSCs on fibronectin-treated glass coverslips treated with 1.5 UM Cytochalasin D for 2 hours and stained with anti-YAP antibody and Hoechst MSC exhibits less nuclear YAP and hence low YAP ratio.

FIG. 11D is a microscopy image showing a 3D Nuclear volumes reconstructed from confocal stacks show swollen nuclei with high nuclear volume.

FIG. 12A is a graph showing nuclear volume decreasing for an increasing YAP ratio (increases to ˜1 for cells in suspension whereas in adherent cells after PEG, nuclear volume decreases, and the YAP Ratio increases to more than 1).

FIG. 12B is a graph showing the nuclear volumes of cells after PEG compression which do not correlate with the YAP ratio (suspended cells below the solid line and adherent cells above the solid line).

FIG. 12C is a graph showing the percentage change in Kmax XZ correlates with the percent change in YAP ratio.

FIG. 12D is a graph showing the percentage change in nuclear volume does not correlate with the percent change in YAP ratio.

FIG. 13A is a microscopy image showing Hoechst-stained nuclei of wildtype MSC in XY, YZ, and XZ projection.

FIG. 13B is a microscopy image showing nuclear curvatures for Hoechst stained nuclei of wildtype MSC in XY, YZ, and XZ projection.

FIG. 13C is a microscopy image showing Hoechst-stained nuclei of Cyto D MSC in XY, YZ, and XZ projection.

FIG. 13D is a microscopy image showing nuclear curvatures for Hoechst stained nuclei of Cyto D MSC in XY, YZ, and XZ projection.

FIG. 13E is a graph showing the YAP ratio in function of the Kmax in XY for wildtype cells.

FIG. 13F is a graph showing the YAP ratio in function of the Kmax in YZ for wildtype cells.

FIG. 14A is a microscopy image of MSCs immunostained with actin antibody and

Hoechst showing actin stress fibers compressing the nucleus laterally with decreasing line pattern thickness.

FIG. 14B is a graph showing that the increase in Kmax XZ increases YAP Ratio.

FIG. 14C is a graph showing that the increase in Kmax YZ increases YAP Ratio.

FIG. 15A is a microscopy image showing wildtype MSCs that shows high nuclear YAP on thin line pattern (1.5 μm) and low nuclear YAP on thick line patterns (8 μm).

FIG. 15B is a microscopy image of MSCs treated with Pitstop2 which shows low nuclear YAP on both thick (8 μm) and thin (1.5 μm) line patterns.

FIG. 15C is a microscopy image of MSCs treated with importazole that shows low nuclear YAP on both thick (8 μm) and thin line patterns (1.5 μm).

FIG. 15D is a microscopy image of MSCs treated with a combination of importazole and trans1-2cyclohexanediol (CHD) that shows low nuclear YAP on both thick (8 μm) and thin line patterns (1.5 μm).

FIG. 15E is a graph showing the YAP ratio in function of Kmax XY for the conditions of FIGS. 15A-15D.

FIG. 16A is a graph of the FRET intensity of MSCs confined on thin line patterns (1.5 μm) in function of the Kmax XY which shows an even distribution of nuclear membrane tension across the nuclear curvature.

FIG. 16B is a graph of the FRET intensity of MSCs confined on thick line fibronectin patterns in function of the Kmax XY that shows an increase in nuclear membrane tension with an increase in nuclear curvature.

FIG. 16C is a FRET microscopy image of a cell in the condition of FIG. 16A.

FIG. 16D is a FRET microscopy image of a cell in the condition of FIG. 16B.

FIG. 17A is a graph of MSCs transfected with Nesprin TS confined on fibronectin-coated glass coverslips were exposed to hyperosmotic pressure with 10% PEG400 and hypoosmotic swelling with 50% water. Nuclear membrane tension increases after both nuclear compression with hyperosmotic pressure and swelling with hypoosmotic pressure.

FIG. 17B is a graph showing MSCs transfected with iRFP (red fluorescent protein) YAP and stained with Hoechst confined on fibronectin-coated glass coverslips were exposed to hyperosmotic pressure with 10% PEG400 and hypoosmotic swelling with 50% water. YAP Ratio increases after nuclear compression with hyperosmotic pressure and decreases after nuclear swelling with hypoosmotic pressure.

FIG. 17C is a graph showing MSCs transfected with iRFP YAP and stained with Hoechst confined on fibronectin-coated glass coverslips were exposed to hyperosmotic pressure with 10% PEG400 and hypoosmotic swelling with 50% water. Maximum nuclear curvature increases after nuclear compression with hyperosmotic pressure and decreases after nuclear swelling with hypoosmotic pressure.

### DETAILED DESCRIPTION

Nuclear mechanosensing of actomyosin contractile forces mechanically regulates gene expression of differentiation markers and signalling proteins. One essential signaling protein is Yes-Associated Protein (YAP), a transcriptional coactivator that localizes from the cytoplasm to the nucleus, where it activates transcriptional enhanced associate domain (TEAD) transcription factors (TF) which express genes related to cell proliferation, anti-apoptosis, and differentiation. In MSCs, YAP localization determines differentiation; elevated YAP nuclear localization results in osteogenic differentiation, whereas cytoplasmic retention of YAP leads to adipogenic differentiation. The Hippo signaling pathway regulates YAP localization via its phosphorylation, however, YAP localization is also regulated by mechanical cues such as substrate stiffness, cytoskeletal contractility, and nuclear deformations, which are independent of the Hippo pathway. Indeed, mechanoregulation of YAP also appears to provide a stronger signal than the Hippo pathway, where inhibiting Hippo pathway does not affect stiffness dependent differentiation response, thus, making it crucial to understand YAP response to nuclear mechanosensing.

The physical relationship between cytoskeletal contractility, nuclear volumes, nuclear curvature, and nuclear membrane tension in regulating the YAP localization in MSCs was investigated herein. It was found that YAP nuclear localization in MSCs is regulated by nuclear membrane bending curvature. Physiologically, this nuclear curvature is regulated by contractility induced tension at the nuclear membrane, which scales with nuclear bending and drives YAP nuclear localization. Using pharmacological manipulation of nuclear transport, it was revealed that nuclear curvature-mediated YAP localization is driven by both active and passive nuclear import mechanisms. Finally, the present experiments have shown that the nuclear curvature determines differentiation fate of MSCs, where MSCs with low nuclear curvature differentiate into adipocytes and MSCs with high nuclear curvature differentiate into osteocytes in a environment with factors favorable to both lineages.

Controlling mesenchymal stem cells (MSCs) differentiation is desirable for their use in therapeutic applications, particularly stem cell therapies. Numerous biophysical and mechanical stimuli influence stem cell fate, however, their relative efficacy and specificity in mechanically directed differentiation had remained unclear. Yes-associated protein (YAP) is one key mechanosensitive protein that controls MSC differentiation. It was presently found that nuclear deformation specifically regulates YAP, and its relationship with mechanical stimuli. Here it was reported that maximum nuclear curvature (Kmax) is one of, if not the most, precise biophysical determinant for YAP mechanotransduction mediated MSC differentiation, and is a relevant standard for stem cell-based therapies. Traction force microscopy and confocal microscopy was used to characterize the causal relationships between contractility and nuclear deformation in regulating YAP activity in MSCs. It was observed that an increase in contractility compresses nuclei anisotropically, where the degree of asymmetric compression increased the bending curvature of the nuclear membrane. The membrane curvature and tension was then examined using thin micropatterned adhesive substrate lines and a FRET-based tension sensor, revealing the direct role of curvature in YAP activity driven by both active and passive nuclear import. Finally, micropatterned lines were used to control nuclear curvature and precisely direct MSC differentiation to adipocyte or osteocyte lineages. The present work illustrates that nuclear curvature subsumes other biophysical aspects to control YAP-mediated differentiation in MSCs and can provide a deterministic solution to some of the challenges in mesenchymal stem cell therapies.

Yes-associated protein (YAP) directs MSC differentiation via its activity in shuttling between the nucleus and cytoplasm. Through the control of the mechanistic biophysics responsible for YAP activity the control of MSC differentiation has been achieved herein. Particularly, nuclear curvature precisely directs YAP mechanotransduction and explains mechanosensing observations ranging from substrate stiffness to geometry; increasing nuclear curvature increases YAP activity in MSCs. Specifying nuclear curvature dictates the differentiation of MSCs, with cells with higher nuclear curvature differentiating into osteocytes and cells with lower nuclear curvature differentiating into adipocytes.

Accordingly, there is provided a method of selectively differentiating mesenchymal stem cells into a first predetermined cell lineage associated with the nuclear localization of Yes-associated protein (YAP) or into a second predetermined cell lineage associated with the cytoplasmic localization of YAP. One example of a first cell lineage associated with the nuclear localization of YAP is the osteocyte lineage. Examples of the second cell lineage associated with the cytoplasmic localization of YAP include the adipocyte lineage and the neuronal lineage. To selectively differentiate the MSCs into the first or second stem cell lineage, the maximum curvature of the nucleus (Kmax) is controlled to be at least 0.5 μm⁻¹ to select for the first predetermined cell lineage or to not exceed 0.5 μm⁻¹ to select for the second stem cell lineage. In addition to controlling the curvature, the MSCs are incubated in a media containing suitable differentiation additives to obtain the first predetermined cell lineage or the second predetermined cell lineage. Examples of media supplements include β-glycerol phosphate, ascorbic acid, and dexamethasone. In preferred embodiments, the Kmax to select for the first stem cell lineage is at least 0.7 μm⁻¹.

Many methods can be used to control the curvature of the nucleus to have a Kmax value in the desired range. One example is physical confinement, which can be combined with some of the other methods described herein. Examples of physical confinement include:
Micropatterning: confining cells on thin-line micropatterns, induces lateral nuclear compression via actin stress fibers and increases nuclear curvature with decreasing pattern width.
Microchannels: using silicone-based microfabrication thin-line channels of decreasing line width can be generated. Cells cultured inside these channels are restricted to these channel dimensions and get compressed, thus increasing the nuclear curvature with decreasing channel line width.
Vertical Nuclear Compression: cells vertically can be compressed vertically such as by using silicone-based microfabricated pillars of variable height or hydrogel pads made of agarose. The cells and their nucleus can be compressed from the top up to a specific height to increase nuclear curvature.

An advantage of using microchannels and vertical nuclear compression techniques is the consistency of the curvature due to restrictions from channel and pillar dimensions. Alternatively, hydrogel pads provide easy access to cell culture media for the cells to grow thus, increasing their chances of survival. This can however be difficult to achieve in silicone-based microfabricated channels.

It is possible to compress the cells by changing the osmolarity of the media. Cells get compressed under hyperosmotic pressure with 7-13% PEG in culture media and swell under hypoosmotic pressure with 40-60% dextrose in culture media. Under hyperosmotic pressure, the water inside the cells flows out, leading to an anisotropic nuclear compression, with large deformation in Z direction and less in the XY plane. This specific way of nuclear deformation increases nuclear curvature in a 2D setting. Under hypoosmotic pressure, the water is forced inside the cells and the nucleus, thus swelling the nucleus and decreasing its curvature.

It is possible to generate electrospun fibers made of poly(ε-caprolactone) or poly(lactic-co-glycolic acid) for example, these electrospun fibers can have variable thickness, functionalized with cell adhesive proteins such as fibronectin. Confining cells on different thickness microfibers induces a lateral nuclear compression via actin stress fibers with decreasing fiber thickness, similar to the micropatterning technique. The major advantage of this technique is its similarity to the cell microenvironment in 3D, where cells migrate on thin collagen or fibronectin fibers in vivo. This is also a preferred method for industrial applications because it simulates the in vivo environment and has the possibility to scale up in large numbers in well-plate formats.

Osmotic compression is also an attractive industrial scale method due to its simplicity and uniform pressure, or vertical nuclear compression with agarose pads or silicone-based microfabricated pillars. Since they offer a major advantage of getting even or the same nuclear curvature for all the cells. This will reduce the variability among the cell population and also provide a possibility of scaling up in large numbers.

In addition to the nuclear curvature, the volume of the nucleus can be controlled to obtain additional selectivity to the first or second cell lineage. The volume of the nucleus can controlled to be 50% less than the physiological volume of the nucleus to select for the first predetermined cell lineage.

The present findings enable broad bioreactor MSC differentiation strategies.

### Example

### Fabrication of Traction Force Microscopy (TFM) Substrates

To measure contractility using Traction Force Microscopy (TFM), polydimethylsiloxane (PDMS) substrates with different stiffnesses were prepared. In brief, PDMS formulations were prepared by mixing part A and B of commercial PDMS (NuSil™ 8100, NuSil Silicone Technologies, Carpinteria, CA) in 1:1 ratio by weight and supplemented with different concentrations of Sylgard 184 PDMS crosslinking agent (Dimethyl, methyl hydrogen siloxane, which contains methyl terminated silicon hydride units) by weight depending on the desired substrate stiffness. The mechanical properties of the PDMS were measured at different crosslinker concentrations using Rheometer (Anton Paar) and the young's moduli was calculated (Table 1). Then, 50 μl of each solution was applied to the clean 22*22 mm no. 1 glass coverslips and cured at 100° C. for 2 hours. For TFM experiments, prepared PDMS substrates were coated with ˜1 μm thick layer of fluorescent fiduciary particles using spin coater (WS-650 Spin Processor, Laurell Technologies) and incubated at 100° C. for 1 hour. These substrates are referred to as TFM substrates.

TABLE 1
Young's moduli for PDMS substrates containing different
concentrations of Sylgard 184 crosslinking agent

| Additional crosslinker | |
|---|---|
| concentration (weight %) | Young's modulus (YM) (kPa) |
| 0.00 | 0.3 ± 0.05 |
| 0.10 | 2.0 ± 0.06 |
| 0.20 | 5.0 ± 0.04 |
| 0.36 | 12.0 ± 0.71 |
| 0.50 | 23.4 ± 1.86 |
| 1.80 | 100.0 ± 2.80 |

### Surface Modification of TFM Substrates

To facilitate cell attachment, fibronectin was covalently bound to TFM substrates by covering the substrates with 100 μg/ml Sulfo-SANPAH (ThermoFisher Scientific) solution in 100 mM 4-(2-hydroxyethyl)-1-piperazineethanesulfonic acid (HEPES) pH 7.4, followed by exposure to UV for 2 minutes. After UV activation, Sulfo-SANPAH solutions were removed and 10 μg/ml fibronectin (Sigma) solution diluted in phosphate buffer saline (PBS) was added on to the samples, followed by overnight incubation at 4° C. Finally, fibronectin solutions were removed, and substrates were rinsed with PBS 3 times and sterilized under UV.

### UV-Based Micropatterning on Glass

Glass coverslips were adhesively micropatterned on 22*22 mm no. 1 with a UV-based patterning system (PRIMO, Alveole Lab, Paris, France). Glass coverslips were incubated with Poly-L-Lysine (PLL, Sigma) solution (5 mg/mL) prepared in 0.1 M HEPES buffer (pH 8.5) for 1 hour at room temperature, followed by rinsing with MiliQ™ water. Positively charged PLL electrostatically adsorbed onto the negatively charged surface of silicone substrates and allowed protein attachment after printing. The coverslips were then incubated with Polyethylene glycol valeric acid (PEG-SVA, Laysan Bio) prepared in 0.1 M HEPES buffer (pH 8.5) for 30 minutes at room temperature, followed by thorough rinsing with phosphate buffer saline (PBS) pH 7. PEG-SVA acts as an antifouling brush layer that repels protein attachment. The coverslips were then covered with the UV sensitive photo-initiator solution of PLPP (Alveole Lab, Paris, France) and placed on the stage of a microscope (Nikon Ti2 Eclipse) equipped with the UV-patterning system.

To generate the patterns, open-source graphics software programs were used, Inkscape and ImageJ, to generate binary 8-bit mask image files that were loaded into PRIMO's control software. The desired pattern was generated by a digital micromirror array in the PRIMO system and projected using a 375 nm UV laser with an intensity of 29 mW/mm² via 20×/0.45NA objective. The projected pattern results in localized photodegradation of the antifouling PEG-SVA brush, in the shape of the desired pattern. An exposure dose of 20 seconds was adequate to complete photodegradation of the PEG-SVA brush. Following UV exposure, the coverslips were washed with PBS and incubated them for 1 hr at room temperature with 40 μg/mL fibronectin (Sigma) in PBS to adsorb the protein to the exposed PLL surface. Excess protein was rinsed off with PBS 3 times prior to cell seeding.

### Cell Culture, Transfections, and Nuclear Staining

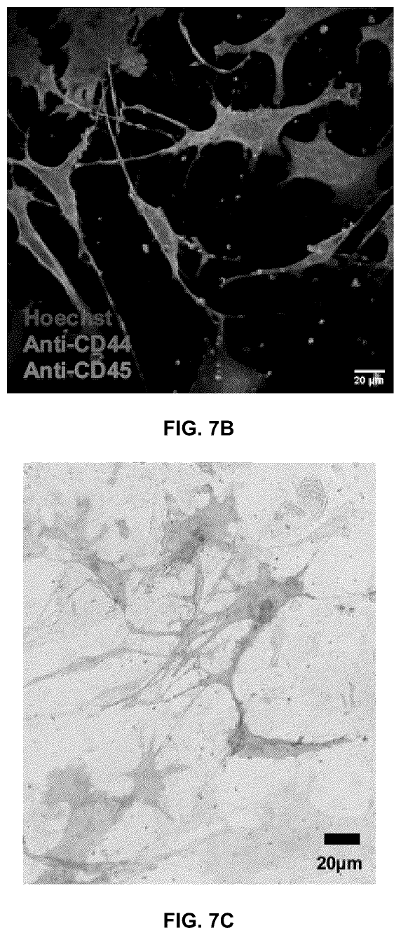

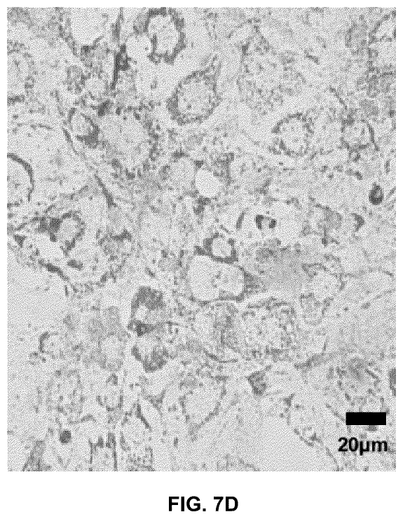

Murine bone marrow stem cell (MSCs) line originated from BALB/c mice (D1s) were purchased from American Type Cell Culture (ATCC) and were cultured in manufacturer specified conditions including standard Dulbecco's modified Eagle medium (DMEM) (Wisent) supplemented with 10% fetal bovine serum (FBS) (Wisent) and 1% Penicillin-Streptomycin antibiotic (P/S) (Thermo Fisher). MSCs were tested according to International Society for Cell and Gene Therapy (ISCT) standards, where first, MSCs were plastic-adherent when maintained in standard culture conditions, second, MSCs expressed CD44 and lacked expression of CD45 surface marker, and third, MSCs differentiated into osteocytes and adipocytes in vitro (FIGS. 7A-7D).

The cells were culture at 37° C. in 5% CO₂ environment and maintained at no greater than 80% confluency and lower than 15 passage number. For TFM and hyperosmotic compression experiments, cells were trypsinized with 0.25% Trypsin (Wisent™), and seeded at a density of 0.2 million cells per ml 16-18 hours before imaging at 37° C. in 5% CO₂ environment. For hyperosmotic compression experiments, suspended cells were achieved by seeding cells on untreated glass coverslips right before imaging and maintained at 37° C. in 5% CO₂ environment while imaging. For Micropatterning experiments, cells were seeded on the micropatterns for 1 hour at 37° C. in 5% CO₂ environment, followed by a gentle wash with PBS to remove non-attached cells to avoid nonspecific attachments. Cells were further incubated for 16-18 hours (on micropatterns) before imaging at 37° C. in 5% CO₂ environment.

To quantify YAP localization and nuclear deformation, MSCs were transiently transfected with iRFP-YAP plasmid (a gift from Xavier Trepat, Institute for Bioengineering of Catalonia (IBEC), Barcelona) and after 48 hours just before imaging cells were stained with 10 μg/ml Bisbenzimide Hoechst 33342 trihydrochloride (Sigma) in PBS for 10 minutes, followed by wash with PBS and addition of complete DMEM media.

To measure nuclear membrane tension, MSCs were co-transfected Nesprin tension sensor (pcdna nesprin TS, 68127, Addgene) and iRFP-YAP plasmid. Nesprin tension sensors are fluorescent resonance energy transfer-based tension biosensors for nesprin 2G, a structural protein at the LINC complex, which links actin cytoskeleton to the nucleus. For control experiments, MSCs were transfected with Nesprin headless control (pcdna nesprin HL, 68128, Addgene), which lacks the actin-binding domain and is localized on the nuclear membrane.

All the plasmids were transfected using GenJet™ Plus transfection reagent (Signagen), as per manufactures instructions. Cells were seeded on the desired samples 18-24 hours after transfections and further incubated for 16-18 hours before imaging at 37° C. in 5% CO₂ environment.

### Confocal Microscopy

For live cell imaging, cells were transferred to a lab-built heated stage perfused with 5% CO₂ and mounted on a confocal microscope (Leica™ TCS SP8, 63× objective). With this setup, live cells were imaged with transmission and fluorescence microscopy for extended periods, while maintaining a controlled culture environment.

### YAP Localization Imaging and Analysis

For YAP localization measurements MSCs were imaged in two channels one for iRFP YAP transfected or Alexa 647 conjugated secondary antibody-stained cells (Ex 647 nm laser) and other for Hoechst-stained nuclei (Ex 405 nm laser). The principal metric for YAP quantification was the ratio of fluorescence of iRFP YAP in the nucleus to iRFP YAP in the cytoplasm and is referred to as the YAP Ratio. Image segmentation was performed using a custom MATLAB code, that segments the nucleus from Hoechst and cytoplasm from YAP and measures the ratio of total iRFP YAP fluorescence inside the nucleus to total iRFP YAP fluorescence in the cytoplasm.

### Immunostaining

To observe endogenous YAP localization, the MSCs were fixed with 4% paraformaldehyde for 15 min in room temperature and washed 3 times with PBS. The cells were permeabilized with 0.1% Triton X-100 diluted in PBS for 10 minutes. To avoid any nonspecific binding, cells were incubated with 1% bovine serum albumin (BSA) in PBS for 30 minutes at room temperature. To visualize YAP, cells were first incubated with 10 μg/ml Anti-YAP1 mouse monoclonal antibody (ab56701, Abcam™) diluted in 1% BSA in PBS overnight, followed by PBS wash and 1 hour incubation with 10 μg/ml Donkey Anti-Mouse IgG H&L (Alexa Fluor™ 647) secondary antibody (ab150107, Abcam™) diluted in 1% BSA in PBS. For nuclear characterization, nuclei were stained with 10 μg/ml Bisbenzimide Hoechst 33342 trihydrochloride (Sigma™) in PBS for 20 minutes, cells were washed with PBS.

### MSC Differentiation

To differentiate stem cells under lateral nuclear compression, first, MSCs were confined on fibronectin line patterns with decreasing thickness (1.5, 2, 4, 6 and 8 μm), as described above. Second, to induce differentiation complete MSC media was supplemented with 10 mM β-glycerol phosphate (Sigma), 50 μg/ml ascorbic acid (Sigma) and 0.1 μM dexamethasone (Sigma). MSCs were differentiated for two weeks, and cultured medium was replaced every alternate day.

For chemically induced differentiation, MSCs medium was supplemented with 10 mM β-glycerol phosphate (Sigma), 50 μg/mL ascorbic acid (Sigma) and 0.1 μM dexamethasone (Sigma) for osteogenesis and only 0.1 μM dexamethasone (Sigma) for adipogenesis.

### Analysis of MSC Lineage Specification

After two weeks in culture, MSCs were fixed with 4% paraformaldehyde for 15 min in room temperature and washed 3 times with PBS. For osteogenesis, alkaline phosphatase activity (ALP, osteogenesis biomarker) was visualized using BCIP/NBT Substrate Kit (SK-5400, Vector Laboratories) as per manufacturer's instructions. For adipogenesis, fat lipid accumulation (Lpdx, adipogenesis marker) was visualized using HCS LipidTOX™ Green Neutral Lipid Stain (H34475, Thermo) as per manufacturer's instructions. For nuclear characterization, nuclei were stained with 10 μg/ml Bisbenzimide Hoechst 33342 trihydrochloride (Sigma) in PBS for 20 minutes, cells were washed with PBS.

The differentiated MSCs were imaged using a confocal microscope (Leica TCS SP8, 63× objective) in three channels, ALP (Ex 647 laser), Lpdx (Ex 488) and Hoechst (Ex 405). The metric that was used to quantify differentiation was a ratio of total fluorescence of ALP to the total fluorescence of Lpdx and is referred to as ALP/Lpdx Ratio. Image segmentation was performed using a custom MATLAB code, that thresholds and generates a cell mask from fluorescence intensity and measures the total fluorescence of ALP and Lpdx for each cell and calculates the ALP/Lpdx Ratio.

### FRET Image Acquisition and Analysis

To measure nuclear membrane tension, MSCs transfected with Nesprin 2G tension sensor were imaged in two channels simultaneously, one for mTFP (Ex. 448) and other for venus (Ex 488) using a confocal microscope (Leica TCS SP8, 63× objective). In each experiment images were captured on the same day, with the gain and laser intensities fixed across all samples.

All the image processing and analysis was performed on open-source Image J software. First, to reduce noise all images for both mTFP and mVenus channels were background subtracted using subtract background with rolling ball radius of 50. Second, the images for both channels were registered using multi-stack registration plugin using mTFP channel as a reference and mVenus image to teal image. Images were spectrally unmixed into mTFP and mVenus channels spectral unmixing. Ratio images were calculated by dividing the unmixed venus channel by the unmixed teal channel. For each image, binary masks were manually created in ImageJ to exclude pixels outside of the nuclear membrane as determined by the mVenus channel. Mean FRET intensity of the nuclear membrane was used to represent the global tension across the nuclear membrane. Local FRET intensity and local curvature were measured using Kappa Curvature Analysis plugin in Image J. In brief, the 2D FRET images were opened in Kappa Curvature Analysis plugin and using a selection tool, points were manually selected across the nuclear membrane and a closed curvature fit was performed. The plugin directly provided the FRET intensity values across the curvature with a desired moving average.

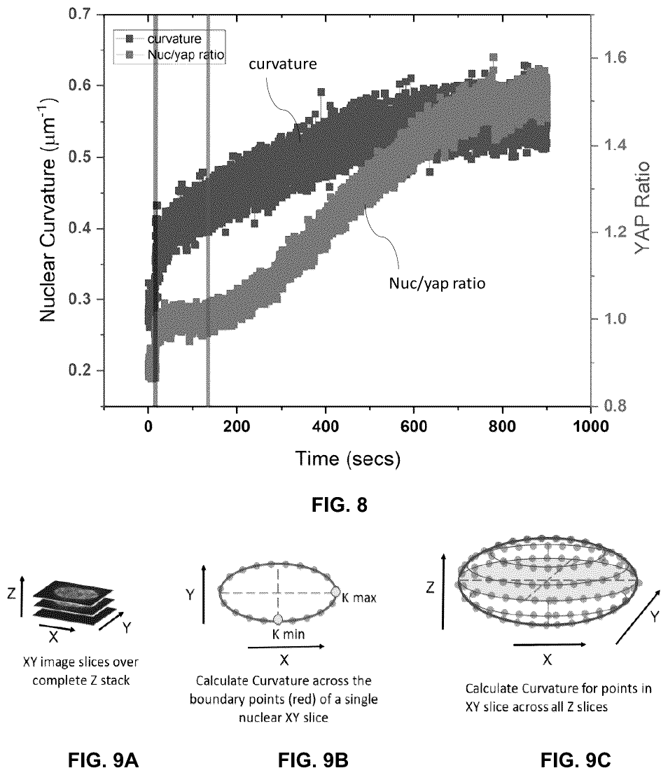

For control experiments, MSCs transfected with Nesprin 2G tension sensor were exposed to cytochalasin D (CytoD), to depolymerize actin network (see pharmacological treatment section) and FRET images were compared before and after CytoD. An increase in FRET intensity was observed after CytoD which indicated a decrease in nuclear membrane tension after actin depolymerization. Further, MSCs were transfected with Nesprin headless control (Nesprin HL), which is the tension sensor lacking actin binding, hence cannot report nuclear membrane tension. Nesprin HL transfected MSCs were confined on fibronectin line patterns with decreasing thickness to induce lateral nuclear compression and high FRET mean intensities was observed across all the nuclei of MSCs confined on variable thickness patterns (FIG. 8). Thus, indicating no change in nuclear membrane tension with increasing nuclear compression.

### Traction Force Microscopy

Cell contractility was quantified using Traction Force Microscopy (TFM) as described above. In brief, MSCs transfected with iRFP YAP (Ex 647) confined on fibronectin coated compliant PDMS substrates of known moduli with a thin layer of fluorescent beads (Ex 552) on top surface were imaged using confocal microscope (Leica™ TCS SP8, 63× objective).

Live MSCs use contractility to deform the underlying substrate which is reflected by displacement of fluorescent beads inside the PDMS substrates. Next, the cells were detached from the substrates surface and captured reference positions of fluorescent beads in the absence of contractility. The average bead displacements of fluorescent beads were calculated by comparing the bead positions with and without cells. The average bead displacements along with the known young's moduli of the PDMS substrates, were then used to calculate total strain energy using Fourier-transform traction cytometry approach. The total strain energy represents the energy applied by the cells to deform the underlying PDMS substrate.

All the image analysis and calculations of average bead displacement and total strain energies were performed using a custom MATLAB code.

### Hyperosmotic Compression

To examine the role of nuclear curvature and nuclear volume in YAP localization, cells were exposed to hyperosmotic pressure in suspended and adherent formats. MSCs transfected with iRFP YAP and stained with Hoechst were seeded on untreated glass coverslips right before imaging for suspended cells and seeded in fibronectin coated glass coverslips 18 hours before imaging for adherent cells. The hyperosmotic pressure of ˜1.62 MPa was applied using 10% 400 Da polyethylene glycol (PEG 400, Sigma) diluted in complete DMEM media. The XY images of iRFP YAP transfected MSCs and XYZ stacks of Hoechst-stained nuclei were captured before and after hyperosmotic compression using a confocal microscope (Leica™ TCS SP8, 63× objective).

### Nuclear Deformation Analysis

To study the role of nuclear deformation in YAP localization, XYZ stacks of Hoechst-stained nuclei of iRFP YAP transfected MSCs were captured using confocal microscope (Leica TCS SP8, 63× objective) with a closed pinhole 0.5 AU and thin Z step size of 0.3 μm. All the XYZ stacks were analyzed using a custom MATLAB code that measures nuclear volumes, surface area, height, major axis, minor axis, and curvature. In brief, the three-dimensional XYZ stacks were, preprocessed by background subtraction threshold, and binarization. The nuclear volumes, surface area, height, major axis, and minor axis were first measured in pixels using regionprops3™ in MATLAB, which were then converted to microns. Nuclear flatness, Nuclear Eccentricity and Nuclear Aspect Ratio were measured using the following equations:

$$\begin{matrix}Flatness = \frac{Semi Major Axis - Semi Nuclear Height}{Semi Major Axis} \\ Eccentricity = \sqrt{1 - \frac{{( \frac{h e i g h t}{2} )}^{2}}{{( \frac{major axis}{2} )}^{2}}} \\ Aspect Ratio = \frac{Major Axis}{Minor Axis}\end{matrix}$$

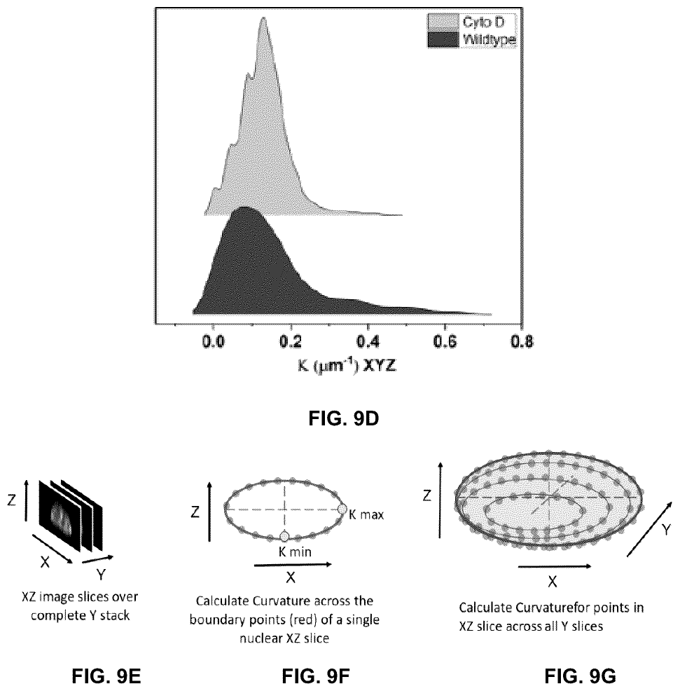

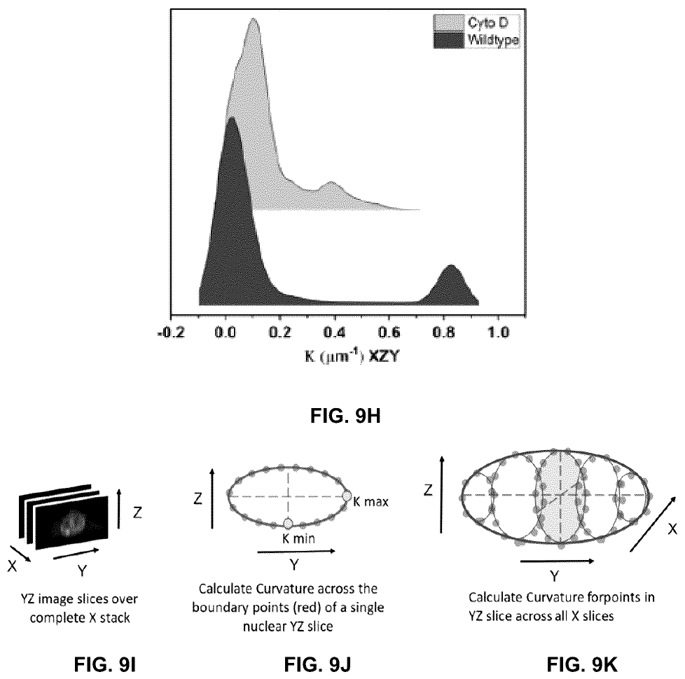

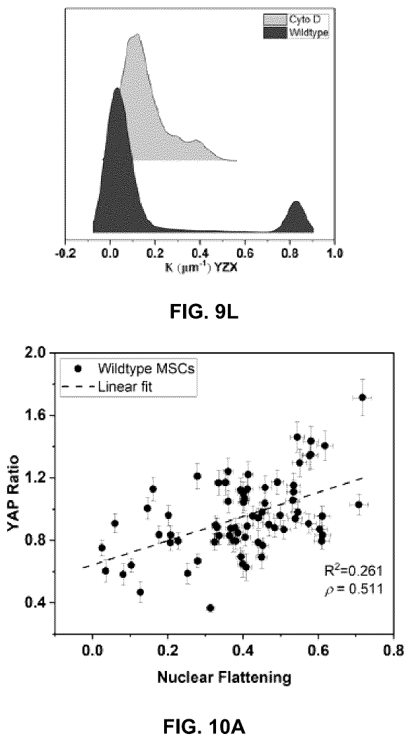

For nuclear curvature analysis, XYZ stacks of Hoechst-stained nuclei were resliced into 2D image sections in XY, XZ and YZ directions. For each resliced 2D nuclear image, curvature was measured across the nuclear boundary, using a curvature function in MATLAB. This step was repeated for all the resliced images in every single dimension. This provided us with distribution of curvature values of the complete nucleus quantified from resliced nuclear images in all three dimensions of XY, XZ and YZ separately (FIGS. 9A-9L).

For control experiments, nuclear curvature of wildtype and Cyto D treated MSCs was measured using XYZ stacks of Hoechst-stained nuclei. For wildtype MSC nuclei, a distribution of high curvature values was observed only for curvature analyzed from XZ and YZ resliced nuclear images (FIGS. 9E-9L). For nuclear images resliced in XY, only a distribution of lower curvature values for the same wildtype MSC nuclei was observed. The distribution of high curvature values observed only in Z resliced images, represents the high local bending of the nuclear membrane due to contractility induced nuclear compression in Z direction. However, lower curvature values from XY resliced images are due to limited nuclear deformation in XY direction, not enough to bend the nuclear membrane. For uncompressed nuclei of Cyto D treated MSCs a similar distribution of low curvature values when analyzed from resliced nuclear images in all the three dimensions (FIGS. 9D, 9H and 9L). Thus, presence of cell contractility in wildtype cells compresses the nuclei and increases nuclear curvature, whereas disrupting the contractility releases nuclear compression and lowers the nuclear curvature.

### Pharmacological Treatments

To confirm the effect of contractility and nuclear deformation on endogenous YAP nuclear localization, MSCs were treated with 1.5 μM Cytochalasin D for 2 hours (CytoD, ThermoFisher Scientific) to depolymerize actin filaments and inhibit the effects of cell contractility on nuclear deformation and YAP localization. After, 2 hours MSCs were fixed and stained with Anti-YAP1 mouse monoclonal antibody (ab56701, Abcam™), Donkey Anti-Mouse IgG H&L (Alexa Fluor™ 647) secondary antibody (ab150107, Abcam™) and Bisbenzimide Hoechst 33342 trihydrochloride (Sigma) for nuclear imaging (see immunostaining section for more detailed protocol).

To identify the mechanisms involved in YAP localization via nuclear curvature, MSCs were confined on fibronectin line patterns of decreasing thickness and then exposed to various drugs that manipulate active and passive nuclear import mechanisms. Finally, MSCs were fixed and stained with Anti-YAP1 antibody and Hoechst as explained above. To inhibit active nuclear import, MSCs were treated with 40 μM Importazole (cayman chemicals) for 1 hour, it inhibits importinβ/Ran GTP nuclear import pathway. For inhibiting both active and passive nuclear import, MSCs were treated with 30 μM Pitstop2 (abcam) for 1 hour, it disrupts nuclear pore complex structure and blocks nuclear pores. To promote passive nuclear import, MSCs were treated with 258 mM trans-1,2-Cyclohexanediol (Sigma) for 5 mins, it disrupts FG nups and dilates nuclear pore complex.

### Contractility Increases Nuclear Compression and YAP Nuclear Localization in MSCs

To determine how cell contractility deforms the nucleus and affects YAP, the contractility, nuclear deformation, and YAP localization in single MSCs were simultaneously measured.

To quantify YAP localization, MSCs were transfected with iRFP YAP and stained with Hoechst to visualize the nucleus. The ratio of the total iRFP YAP fluorescence intensity in the nucleus to that in the cytoplasm (henceforth “YAP Ratio”) was used to quantify iRFP YAP localization in MSCs. The iRFP YAP transfected MSCs were confined on fibronectin-coated soft elastic silicone substrates with fluorescent fiduciary markers to quantify total strain energy using traction force microscopy (TFM). The total strain energy is the total work the cells exert in deforming the underlying substrate via their actomyosin contractility. Finally, three-dimensional image stacks of stained nuclei were captured using confocal microscopy and nuclear volumes, surface area, flatness, heights, and aspect ratio, were measured as different metrics for nuclear deformation.

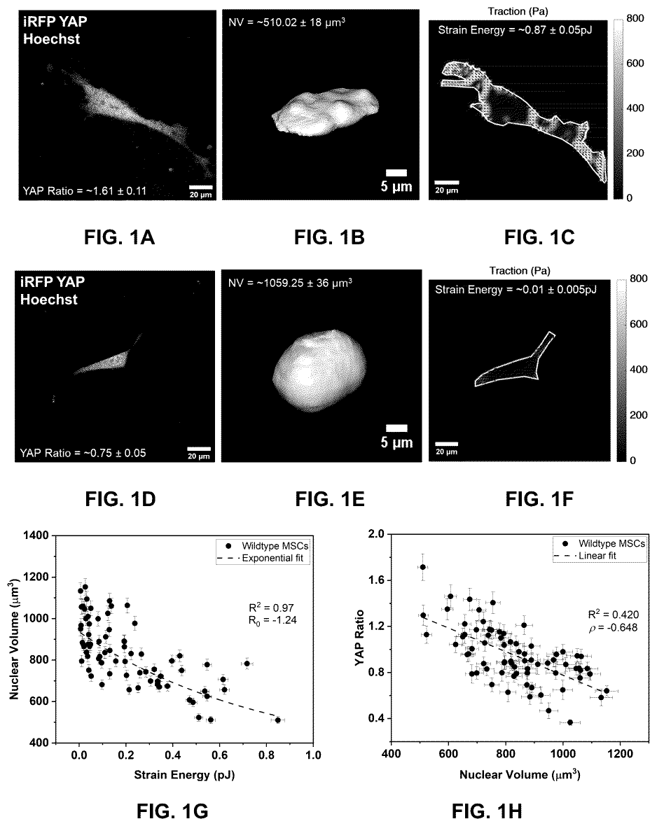

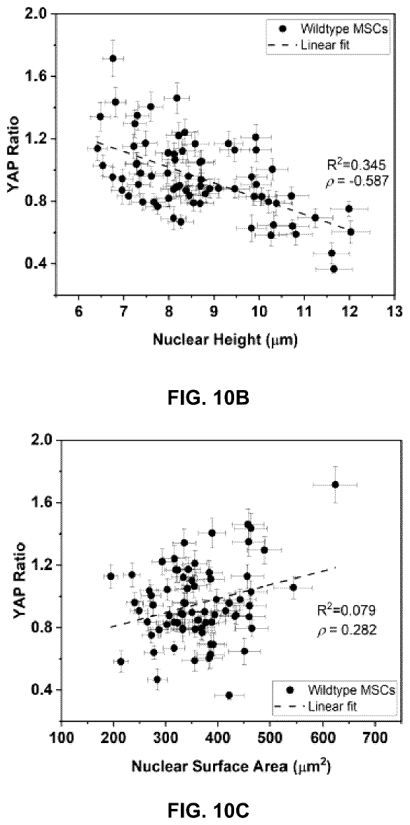

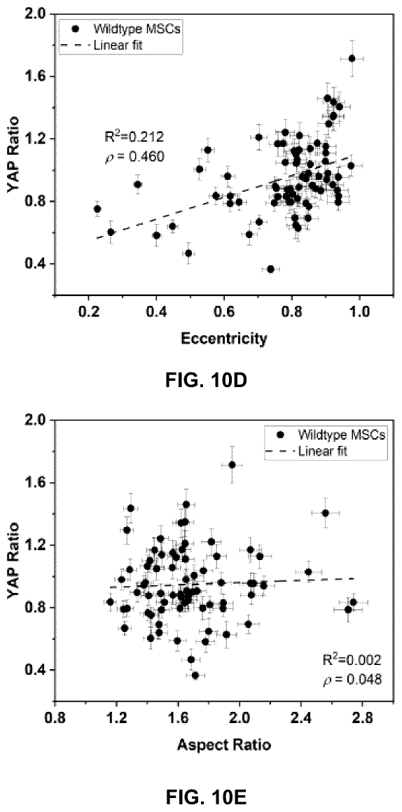

It was observed that MSCs with high strain energy had flatter nuclei with smaller nuclear volumes and more nuclear iRFP YAP, whereas MSCs with low strain energy had rounder nuclei with larger nuclear volumes and less nuclear iRFP YAP (FIGS. 1A-1F). A direct comparison showed us that increasing cell strain energy decreases nuclear volumes (FIG. 1G) and decreasing nuclear volumes increases YAP Ratio (FIG. 1H) suggesting that contractility compresses the nuclei and increases iRFP YAP nuclear localization in MSCs. Further nuclear deformation analysis revealed the nuclear volume correlates the most with iRFP YAP nuclear localization in comparison with nuclear height, flatness, surface area, aspect ratio and eccentricity (FIGS. 1H and 10A-10E).

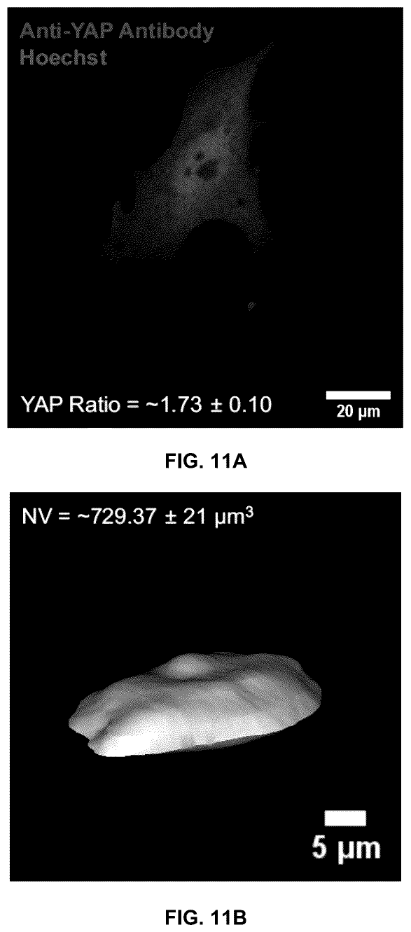

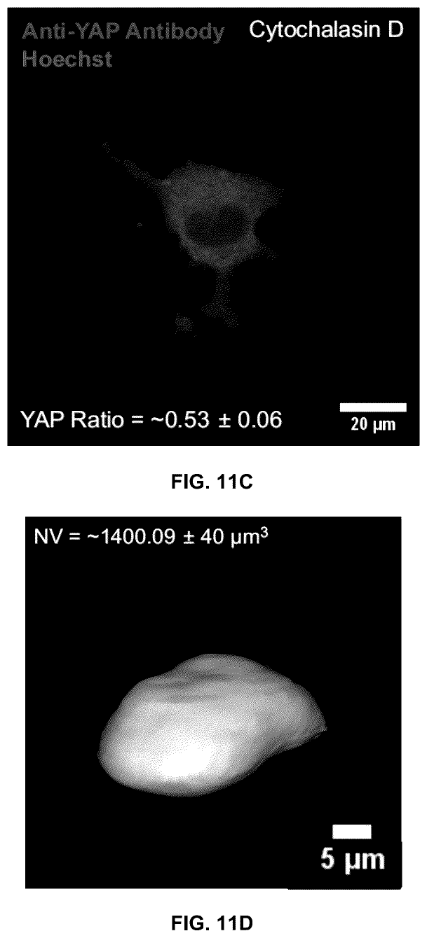

To further examine the causal role of contractility and nuclear compression on endogenous YAP nuclear localization MSCs were cultured on fibronectin coated glass coverslips and the cells were treated with Cytochalasin D (Cyto D) to depolymerize actin network and inhibit contractility. After Cyto D treatment, MSCs were immunostained with Anti-YAP antibody to observe the endogenous YAP localization and stained with Hoechst to visualize the nucleus via confocal microscopy. The wildtype MSCs showed higher YAP ratios with lower nuclear volumes while the Cyto D treated MSCs exhibited lower YAP ratios with high nuclear volumes (FIGS. 11A-11D), further supporting actomyosin contractility dependent nuclear compression driving YAP nuclear localization in MSCs.

Although actomyosin contractility drives nuclear compression physiologically, nuclear compression independent of contractility can drive YAP nuclear localization, where, nuclear compression in contractility inhibited cells increases nuclear YAP. Thus, contractility is not required per se for YAP nuclear localization, as nuclear compression directly regulates YAP. Nevertheless, how exactly the nuclear compression regulates YAP is still unclear.

It was previously understood, the force directly applied to the nucleus in epithelial cells causes nuclear flattening, resulting in nuclear membrane stretch that opens nuclear pores, and increases YAP nuclear localization. The present example has demonstrated that iRFP YAP nuclear localization linearly increases with increasing nuclear flatness (FIGS. 10A-10E), but more strongly correlates with decreasing nuclear volume (FIG. 1H). In a two-dimensional setting, the nuclear flattening represents anisotropic nuclear deformation, with large vertical compression and small lateral deformation. While the anisotropic nuclear compression flattens the nuclei, it can also stretch and bend the nuclear membrane. Since, nuclear localization of any protein including YAP is regulated by physical opening nuclear pore complex (NPC) on the nuclear membrane, both stretching and bending can affect the size or distribution of nuclear pore complex and determine YAP nuclear localization in MSCs. In order to resolve the relative importance of nuclear membrane stretch and bending, both were quantified by measuring nuclear membrane curvature and tension, and compare it with iRFP YAP localization in MSCs.

### Contractility Driven Nuclear Compression Increases Nuclear Curvature and YAP Nuclear Localization in MSCs

First, nuclear membrane bending was quantified by measuring the nuclear curvature (FIGS. 9A-9L). In brief, three-dimensional nuclear stacks captured using confocal microscopy were sliced into two-dimensional image sections in all the three directions (XYZ, XZY and YZX). Then for each two-dimensional nuclear image the curvature across the nuclear boundary points was measured and this step was repeated for all the nuclear image slices in all the three directions. This provided a complete three-dimensional distribution of nuclear curvature values of the whole nucleus, from which the nuclear curvature (K) is obtained. The maximum nuclear curvature (Kmax) is reported herein which represents the maximum bending of the nuclear membrane. The maximum nuclear curvature for the nucleus sliced from all the three directions (Kmax XY, XZ and YZ) was quantified and compared with strain energy and iRFP YAP localization in MSCs.

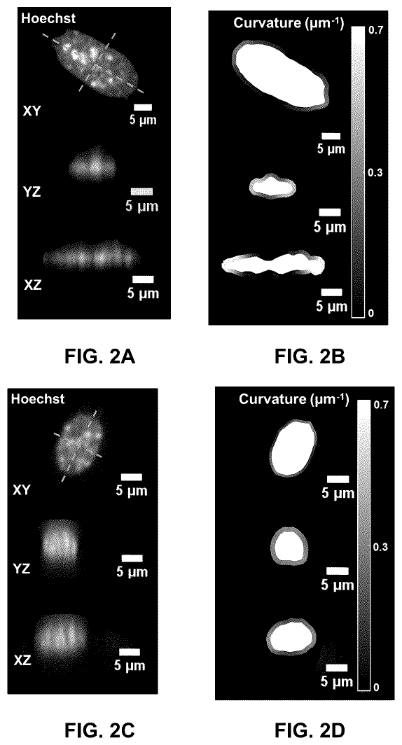

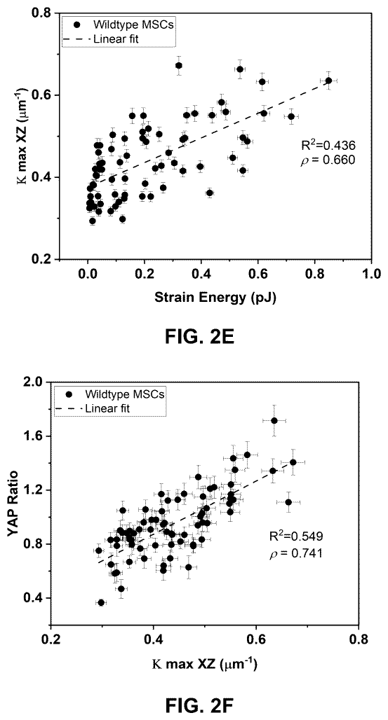

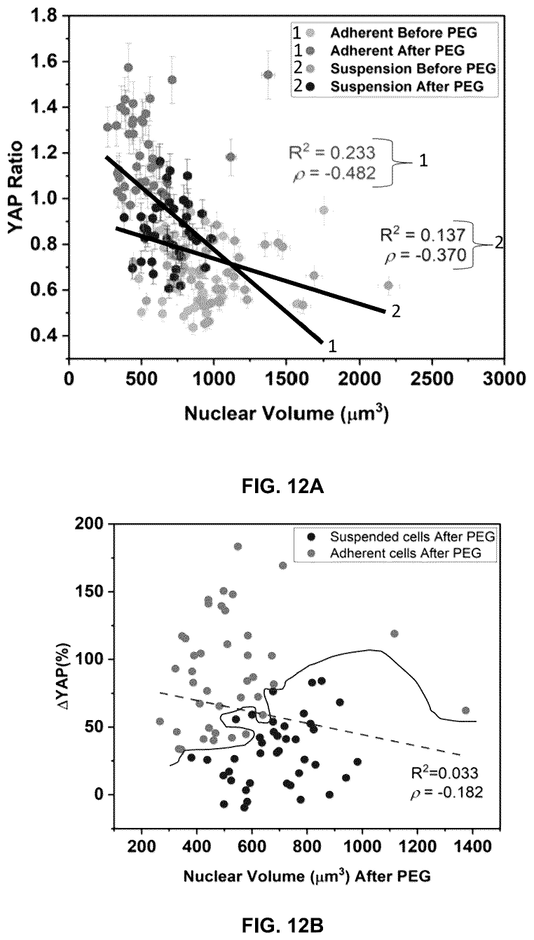

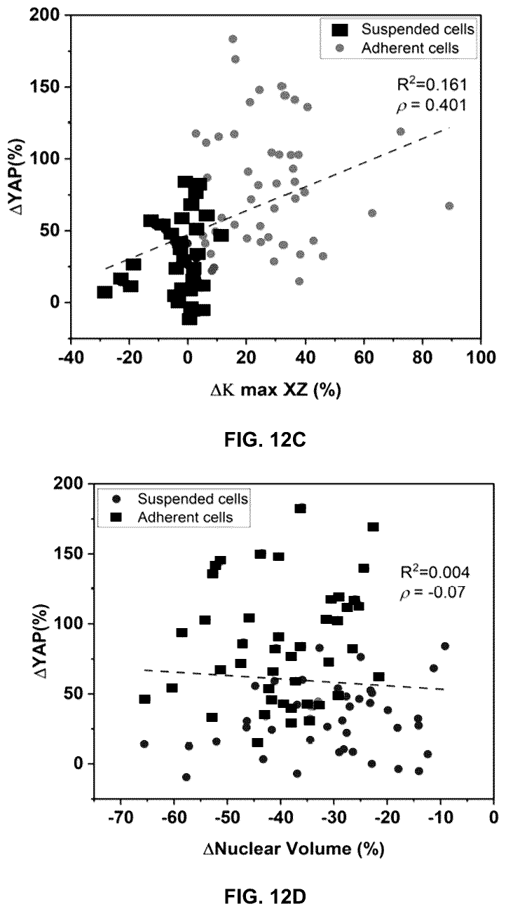

MSCs with high strain energy exhibited flat nuclei with high Kmax in XZ and YZ, whereas MSCs with low strain energy had rounder nuclei with low Kmax in XZ and YZ (FIGS. 2A-2D). There were no pronounced differences in Kmax XY for high and low strain energy MSCs. Further, increased cell strain energy increased Kmax XZ (FIG. 2E), increasing iRFP YAP Ratio (FIG. 2F). When the iRFP YAP Ratio was compared with Kmax measured for the nucleus sliced in other dimensions, it was found that Kmax XZ strongly correlated with iRFP YAP Ratio compared to Kmax XY and YZ (FIGS. 2F, and 12A-12D).

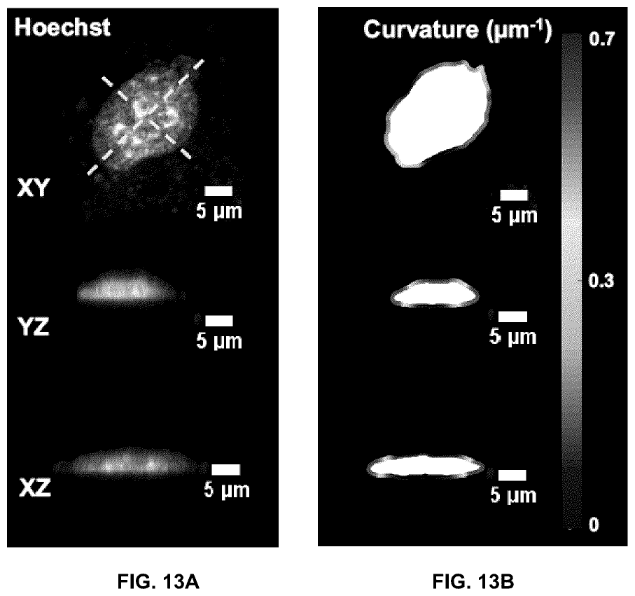

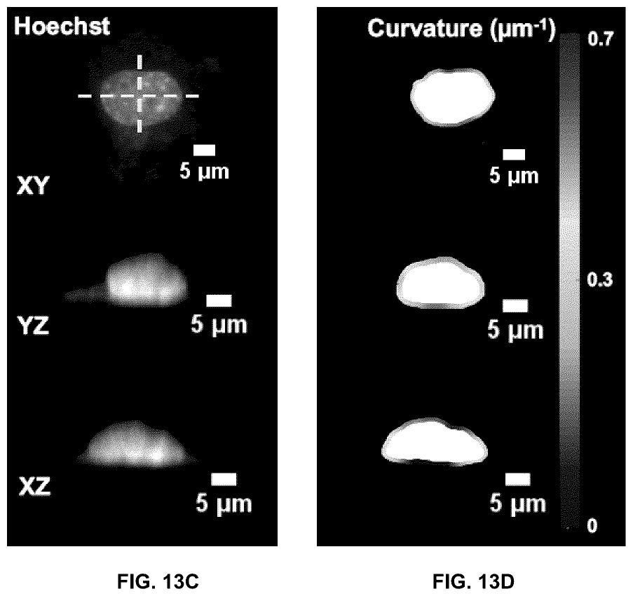

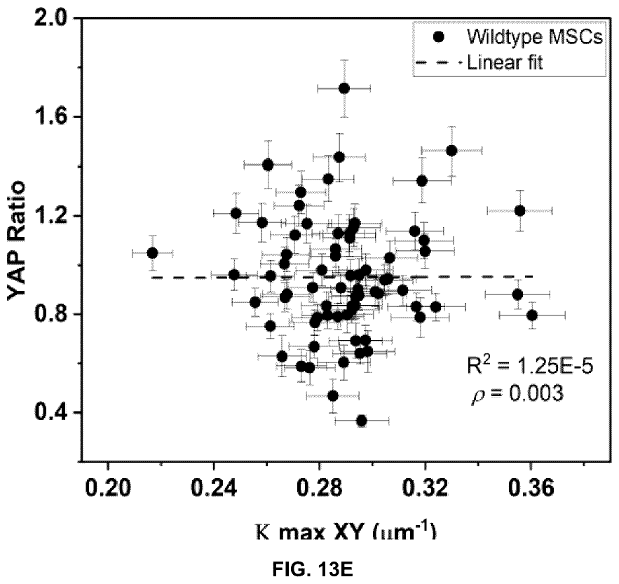

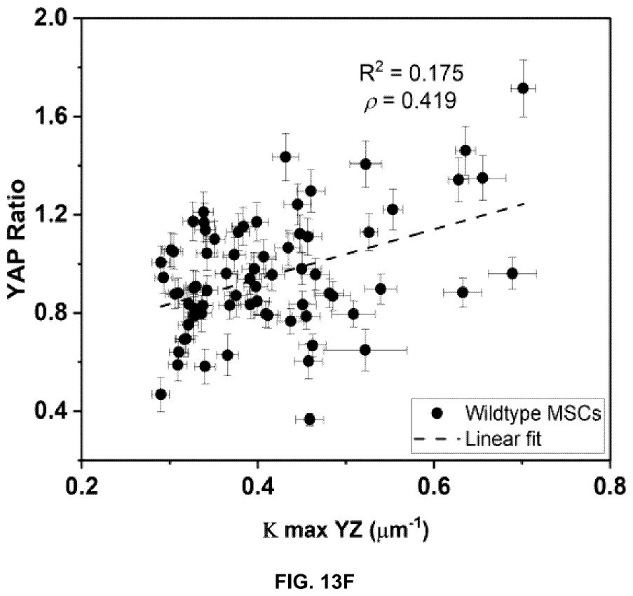

Similar results were observed with endogenous YAP where wildtype MSCs immunostained with Anti-YAP antibody and Hoechst on fibronectin coated glass coverslips presented nuclei with high maximum curvature in XZ and high YAP Ratios; Cyto D treated MSCs exhibited lower maximum curvature in XZ and lower YAP Ratios (FIGS. 13A-13F), further supporting nuclear membrane bending or curvature driving YAP nuclear localization in MSCs.

### Hyperosmotic Pressure Reveals Anisotropic Nuclear Compression Increases Nuclear Curvature and YAP Nuclear Localization in MSCs.

To parse effects of nuclear volume and curvature in determining YAP nuclear localization, iRFP YAP transfected and Hoechst-stained MSCs were exposed to hyperosmotic pressure as adherent and suspended cells. The nuclear volumes, maximum nuclear curvature, and iRFP YAP localization were measured in both adherent and suspended MSCs before and after application of hyperosmotic pressure.

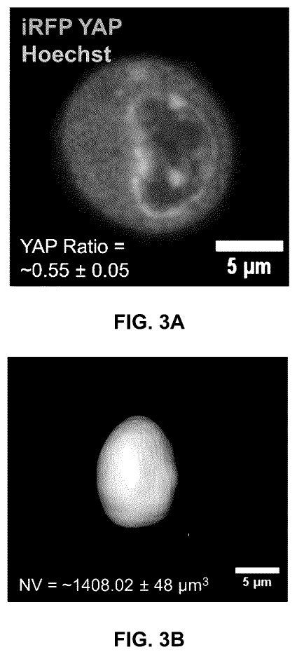

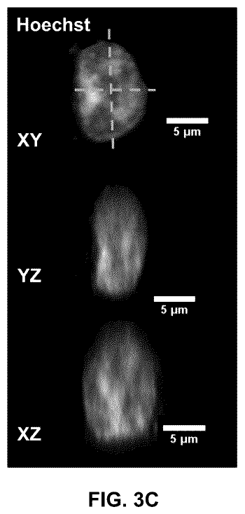

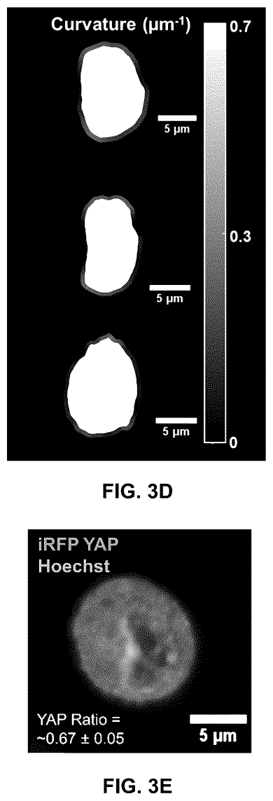

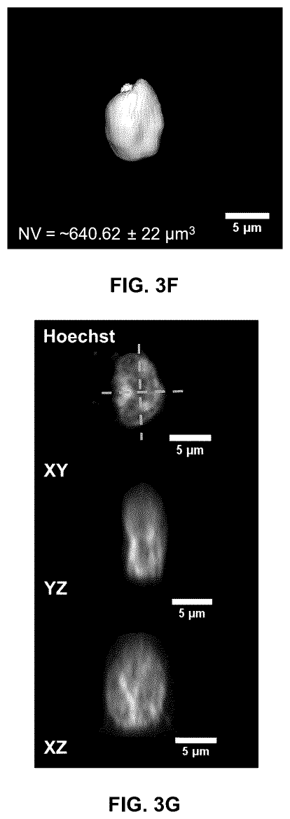

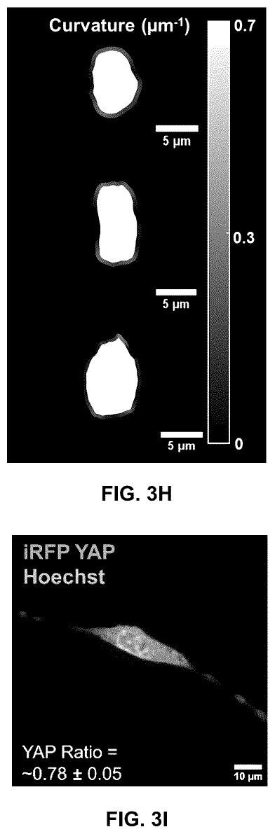

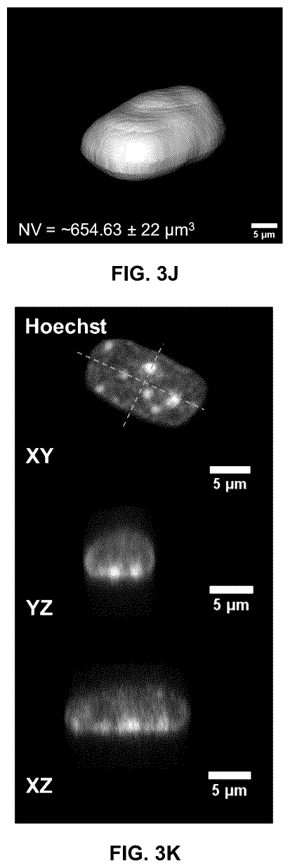

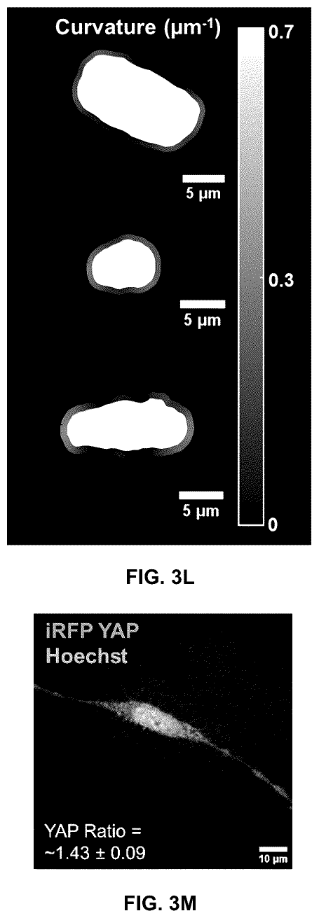

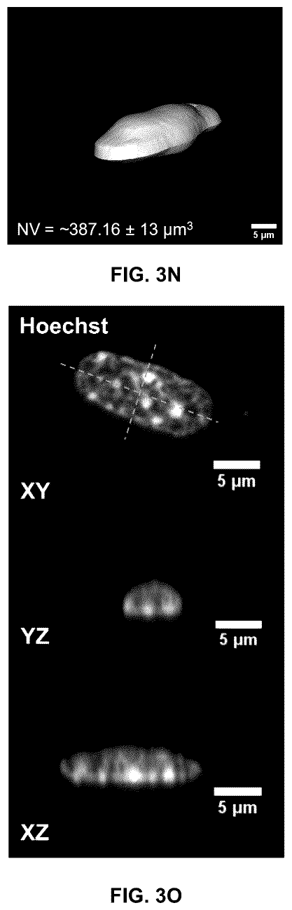

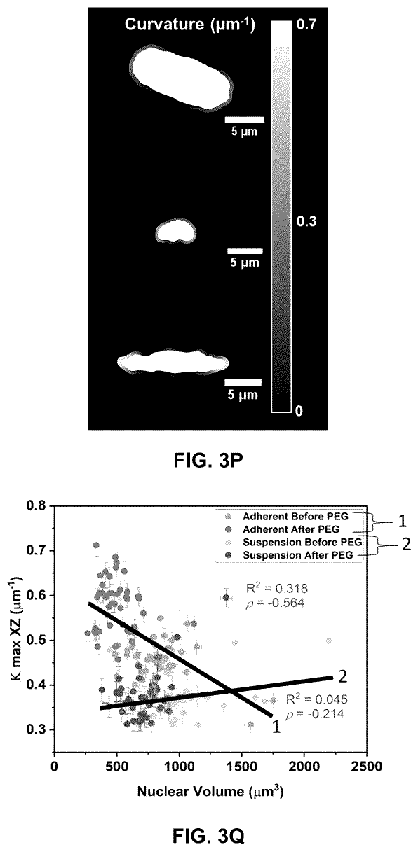

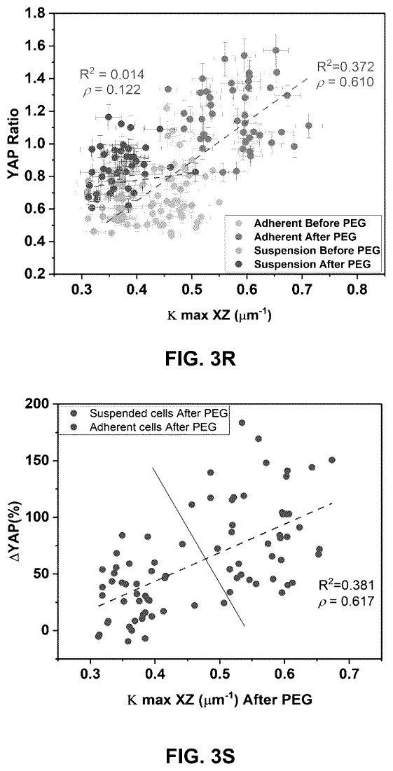

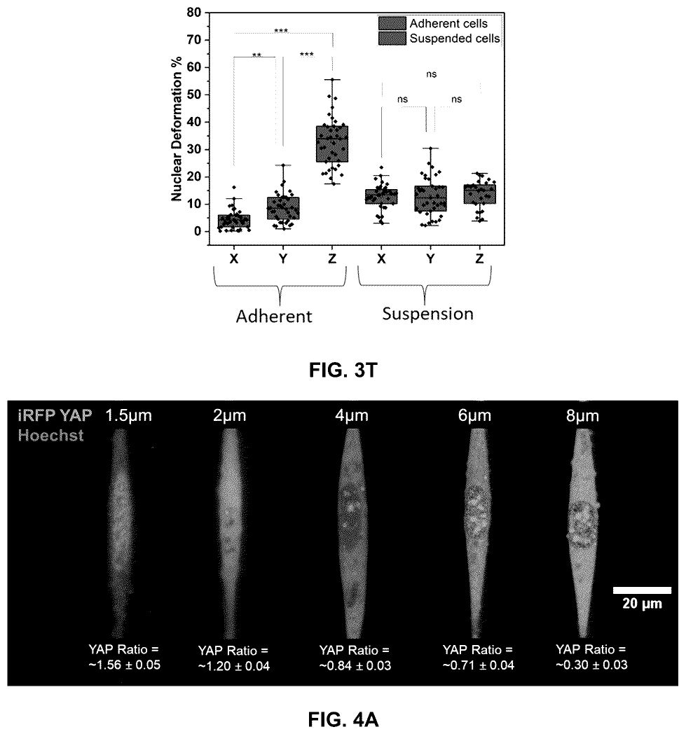

In suspended MSCs, hyperosmotic pressure compressed nuclear volumes isotropically, and thus without evident nuclear membrane bending and iRFP YAP nuclear localization (FIGS. 3A-3H, and 3T). However, in adherent MSCs, hyperosmotic pressure anisotropically compressed nuclear volumes, causing significant increase in nuclear membrane curvature and iRFP YAP nuclear localization (FIGS. 3I-3P, and 3T). A direct comparison between nuclear volumes and maximum nuclear curvature in suspended cells showed no change in Kmax XZ with decreasing nuclear volumes, whereas in adherent cells Kmax XZ increased with decreasing nuclear volumes (FIG. 3Q). Critically, while both suspended and adherent cells had similar nuclear compression, only the adherent cells displayed visible changes in iRFP YAP nuclear localization, concordant with changes in Kmax XZ (FIG. 3R), thus indicating Kmax XZ to be the dominant predictor of iRFP YAP nuclear translocation. This suggests that compression driven YAP mechanosensing only occurs in cells undergoing asymmetric nuclear deformation due some external boundary such as a substrate.

To further clarify the role of nuclear curvature in YAP localization, the nuclear curvature (Kmax XZ), and the change in nuclear curvature (ΔKmax XZ) were compared with the change in YAP Ratio (ΔYAP Ratio). Interestingly, it was observed that increases in Kmax XZ and delta Kmax XZ correlated with increasing ΔYAP Ratio (FIGS. 3S, and 11A-11D). This suggests that the YAP nuclear localization can be determined by the state of the nuclear curvature, and by percent change in the nuclear curvature. Thus, a larger change in nuclear curvature will increase YAP nuclear localization more. Such changes in the YAP Ratio were not affected by the state of nuclear volumes or change in nuclear volumes after hyperosmotic pressure (FIGS. 11A-11D), further evincing the dominant role of nuclear curvature.

Although osmotic pressure compresses the nuclei and increases YAP nuclear localization, it is possible that changing osmolarity itself can influence YAP phosphorylation, which can affect YAP nuclear localization. This makes it crucial to deform nuclei in controlled ways which avoid potential biochemical activation of YAP and provide another strategy to quantify the differences in YAP localization as a function of degree of nuclear compression.

### Nuclear Membrane Bending Determines YAP Nuclear Localization in MSCs.

While osmotically stressing an adherent cell anisotropically compresses the nucleus vertically, it is possible to anisotropically compress the nucleus laterally by confining adherent cells on adhesive line patterns of variable thickness. This technique induces lateral compression of the nucleus via actin stress fibers which also induces a tight local bending of the nuclear membrane, creating variable degrees of nuclear curvature in a controlled manner.

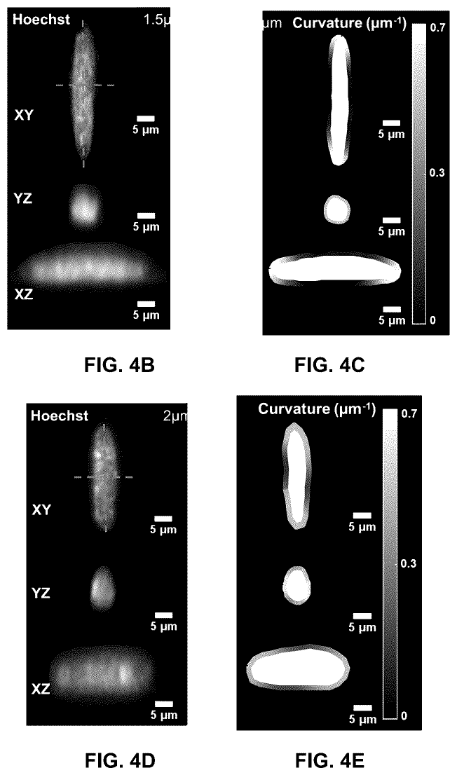

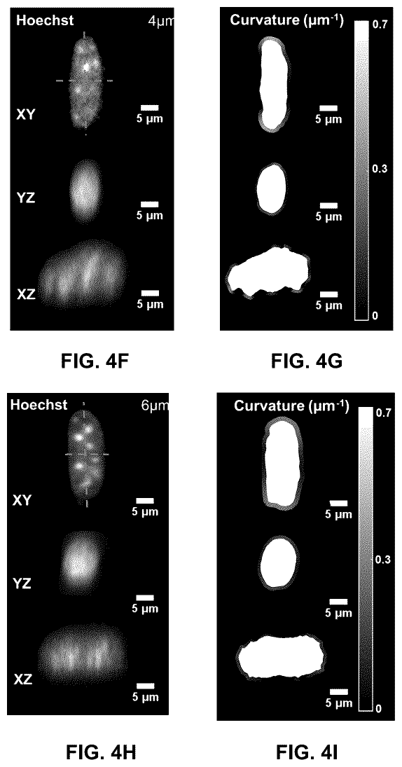

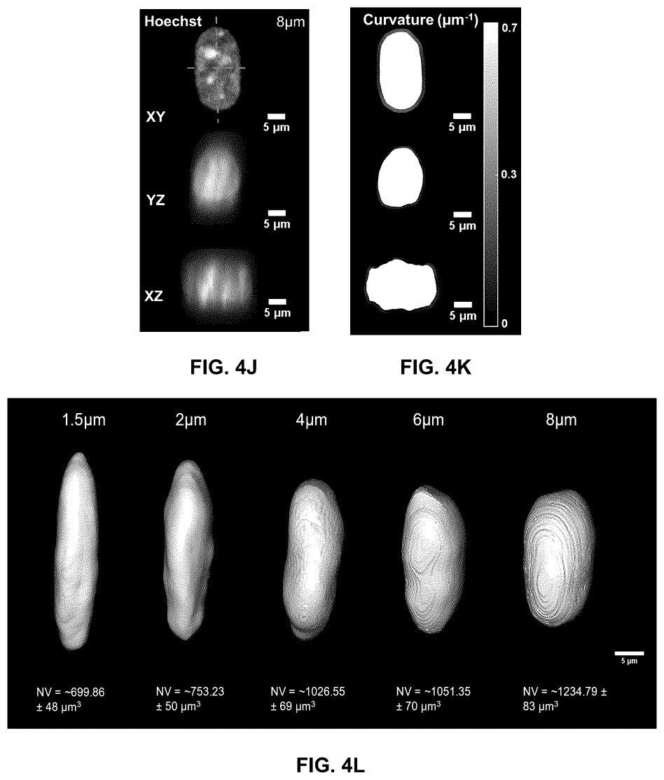

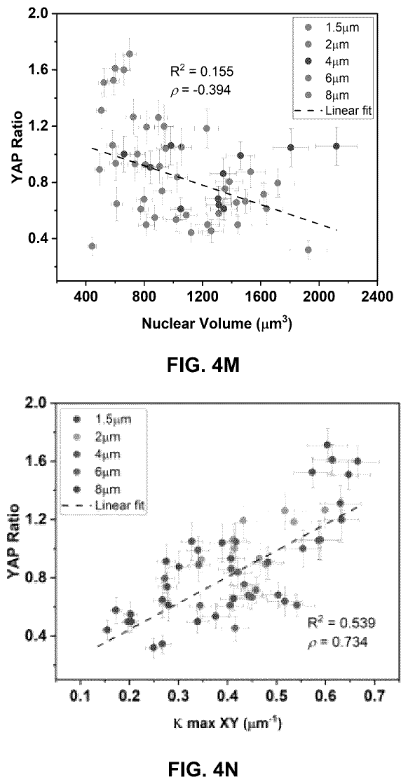

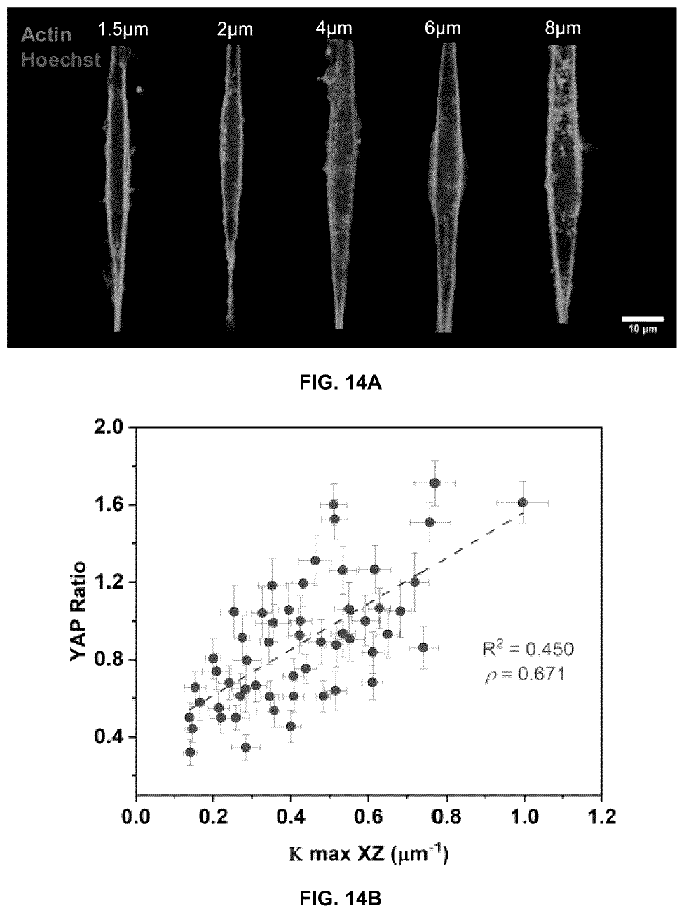

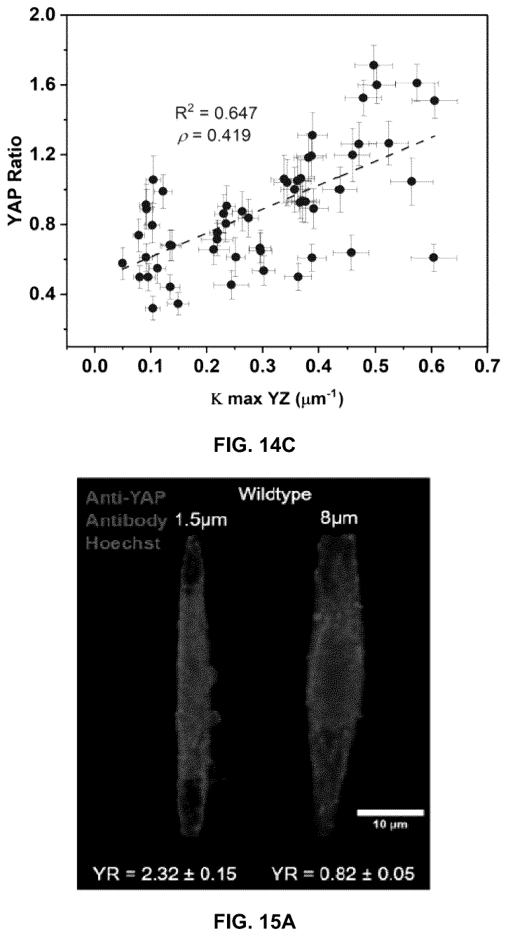

This principle was employed by seeding MSCs transfected with iRFP YAP on fibronectin line micropatterns of variable widths (1.5, 2, 4, 6 and 8 μm), and subsequently stained them with Hoechst nuclear dye. Lateral compression of nuclei via actin stress fibres increased with decreasing pattern width (FIGS. 4B-4L, 14A-14C). Measuring maximum nuclear curvature, nuclear volumes, and YAP ratios for the cells on patterns, it was found that high YAP Ratios in cells confined on narrowest patterns (1.5 and 2 μm, FIG. 4A) with relatively low nuclear volumes and high nuclear curvature (FIG. 4L). However, MSCs confined on 8 μm to 4 μm wide patterns had no notable differences in nuclear volume despite an increase in YAP ratio and nuclear curvature with decreasing pattern width (FIGS. 4A-4L). This presents a divergence in the relationship between nuclear volume and YAP localization, implying curvature plays a more significant role.

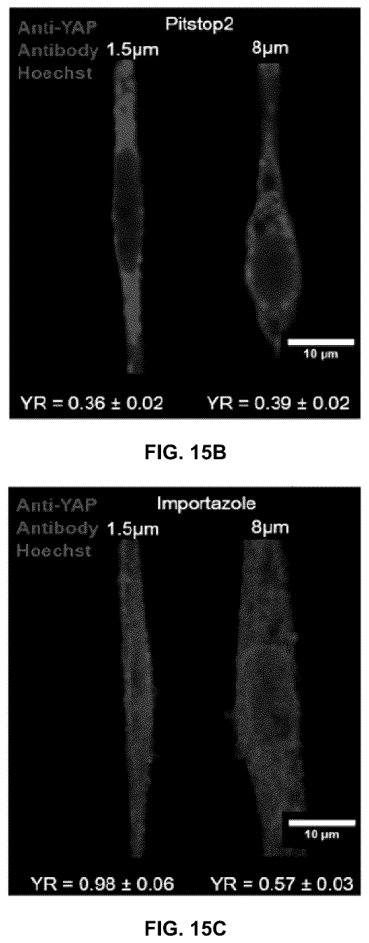

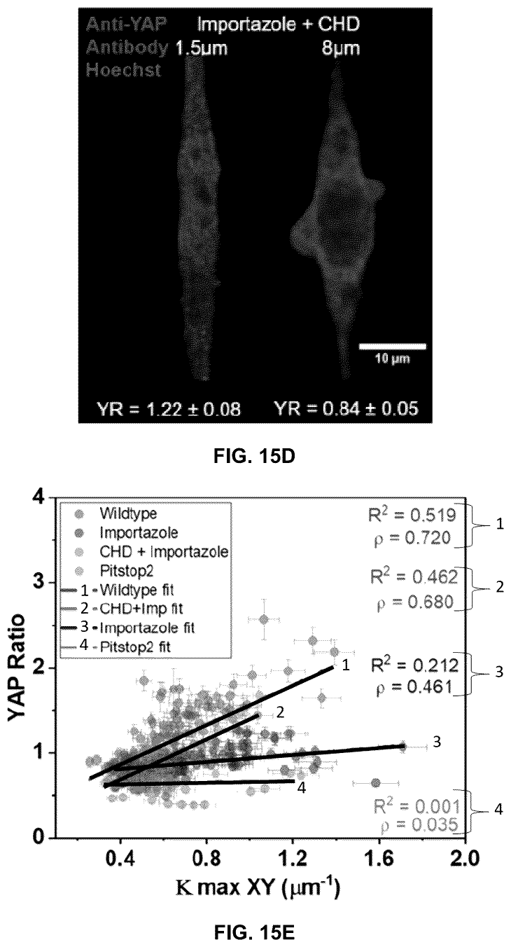

To observe how nuclear curvature influences YAP localization on line patterns, the correlation of the YAP ratio with maximum nuclear curvature as well as nuclear volumes was measured. Increased YAP Ratios were observed with increasing Kmax XY, Kmax XZ and Kmax YZ (FIGS. 4B-K, 14A-14C), with the highest correlation of YAP Ratio with Kmax XY, the plane of lateral compression (FIGS. 4M and 4N). To confirm the effects of nuclear curvature on endogenous YAP localization quantitative immunofluorescence of YAP wildtype MSCs confined on line patterns was performed and again YAP nuclear localization was observed with increasing nuclear curvature (FIGS. 15A-15E). Hence, it was concluded that the local bending of the nuclear membrane induced by the nuclear compression dominates YAP nuclear localization in MSCs. Nevertheless, how nuclear curvature mechanistically influences YAP localization remains unclear.

YAP enters the nucleus in two ways: it is either actively transported inside the nucleus or it can passively diffuse inside the nucleus via opening of nuclear pore complex. It was hypothesized that the increase in nuclear curvature would stretch the nuclear membrane and facilitate the passive diffusion of YAP via opening of nuclear pores. To test this, MSCs were confined again on thin line patterns and treated with pharmacological modulators of nuclear import mechanisms (FIGS. 15A-15E). MSCs were first treated with Pitstop2, which completely disrupts the nuclear pore complex and blocks both active and passive nuclear import. With Pitstop2 a complete inhibition of curvature influenced YAP Ratio was observed, suggesting Kmax XY curvature influences YAP via passive and active nuclear import mechanisms. Next, MSCs were treated with importazole which blocks RAN GTP dependent active nuclear import; a small increase in YAP ratios with increasing Kmax XY was observed, however YAP ratios were substantially less than untreated MSCs, and correlated less with Kmax XY. To test the role of passive nuclear import via nuclear pores, MSCs were treated with a combination of trans1-2 cyclohexanediol (CHD), which dilates pores and increases passive transport, and importazol. CHD functions by disrupting nuclear pore complex Phenylalanine-glycine repeat proteins (FG nups) which act as a mechanical barrier to passive diffusion of proteins, dilating the nuclear pores and thus promoting passive nuclear import. An increase in YAP Ratio was observed with increasing Kmax XY suggesting passive import of YAP with increasing nuclear curvature. However, the YAP Ratio remained lower than the untreated MSCs, which might be due to the blocked active nuclear import via importazole. This suggests complementary and likely redundant roles of both active and passive nuclear import mechanisms in regulating YAP nuclear localization with increasing nuclear curvature.

Nuclear membrane bending can also stretch the nuclear membrane and it is crucial to understand the effects of both nuclear membrane stretch and bending on nuclear localization of YAP.

### Nuclear Membrane Tension Determines Nuclear Curvature and YAP Localization in MSCs

To examine the role of nuclear membrane stretch Forster Resonance Energy Transfer (FRET) based tension sensors was used, which reflects tension across the nuclear membrane. Nesprin 2G tension sensor, is a nesprin (LINC complex protein) based tension sensor that links the actin cytoskeletal network to the nucleus via the SUN binding domain and directly reports mechanical tension applied by actin contractility to the nucleus via LINC complex. For these experiments, MSCs were co-transfected with Nesprin 2G tension sensors and iRFP YAP, and then induced lateral nuclear compression by again confining them on fibronectin lines of decreasing pattern widths as described in the previous sections. Mean intensities of FRET ratio images of the nuclear membrane were measured, along with nuclear curvature, and YAP localization with decreasing pattern widths. For Nesprin 2G tension sensors, a decrease in FRET mean intensity represents an increase in nuclear membrane tension and vice versa.

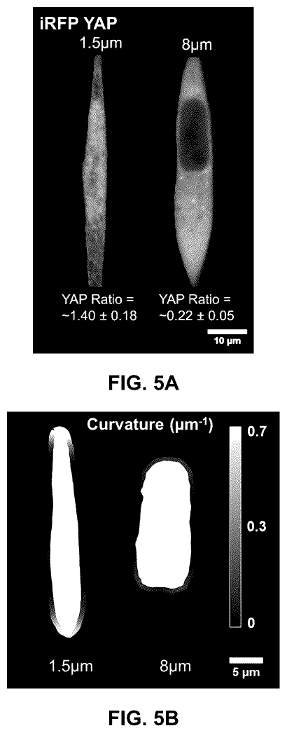

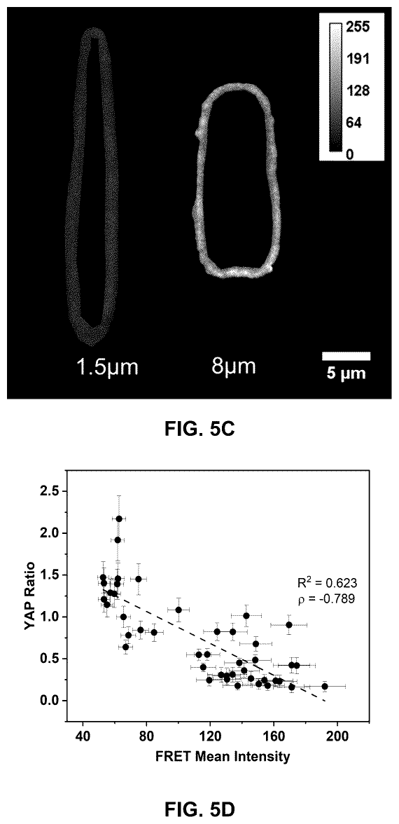

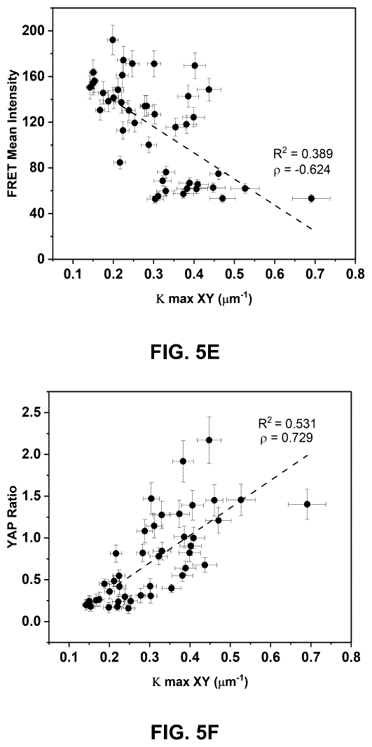

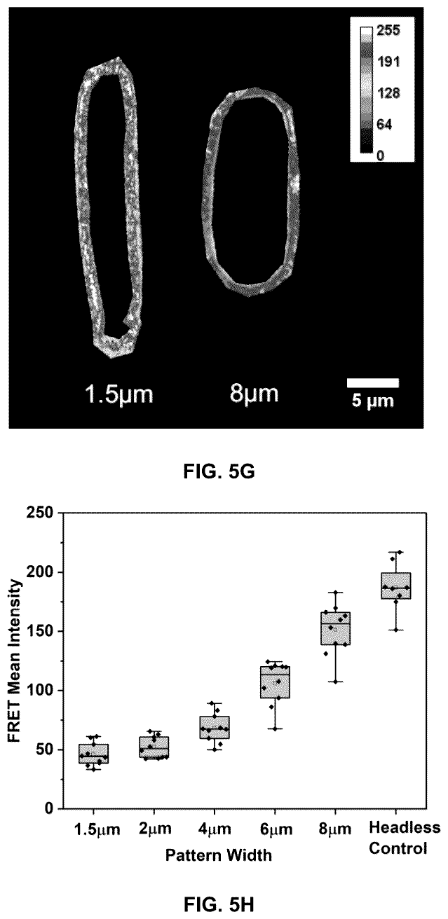

MSCs confined on narrow patterns of 1.5 μm had more nuclear YAP with high nuclear curvature and higher nuclear membrane tension (low FRET mean intensity) (FIGS. 5A, 5B, and 5C). Conversely, MSCs confined on wider patterns of 8 μm had less nuclear YAP with low nuclear curvature and nuclear membrane tension (high FRET mean intensity) (FIGS. 5A, 5B, and 5C). In direct comparisons, it was observed that increasing nuclear membrane tension (decreasing FRET mean intensity) increases YAP Ratio (FIG. 5D). Also, the increase in Kmax XY increases nuclear membrane tension and YAP Ratio (FIGS. 5E and 5F). For control measurements, MSCs transfected with Nesprin headless control on thin and thick fibronectin line patterns and observed low nuclear membrane tension (high FRET mean intensity) in cells on both 8 and 1.5 μm patterns (FIGS. 5G-5H).

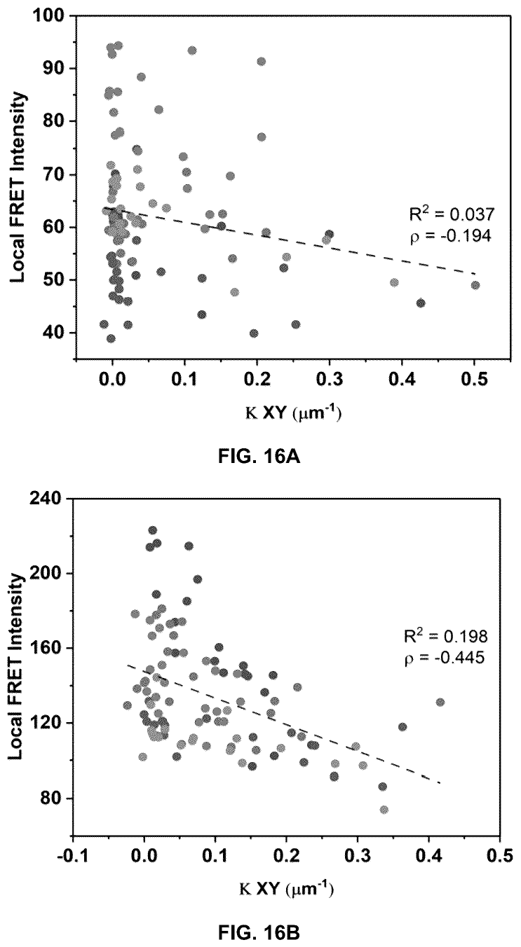

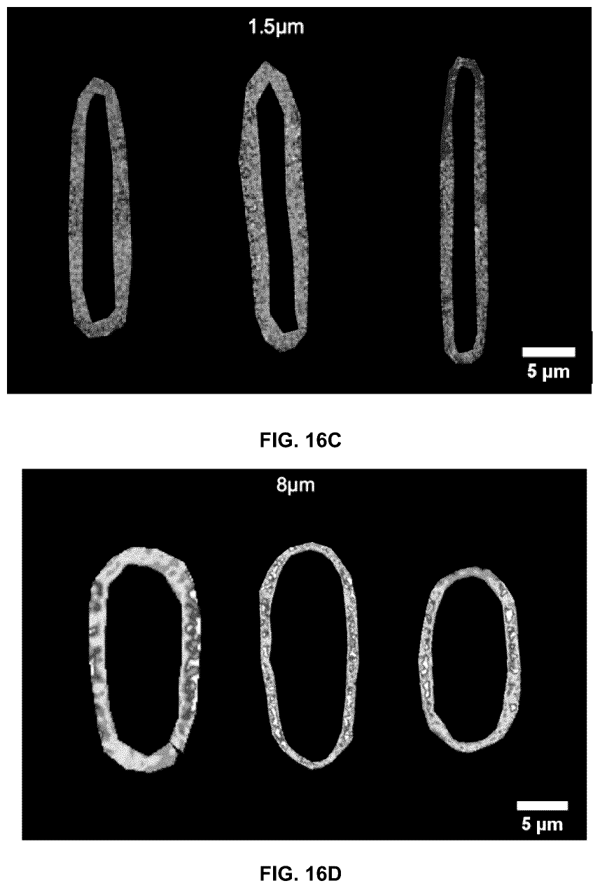

The local tension across the nuclear membrane was also measured and compared with the local curvature (FIGS. 16A-16D). Interestingly, in nuclei of MSCs on wider patterns of 8 μm, an increase in local nuclear membrane tension was observed with increasing local nuclear curvature, but in nuclei of MSCs on thinner patterns of 1.5 μm, it was found that no correlation between local nuclear membrane tension and local nuclear curvature (FIGS. 16A-16D). It is believed that this might be because the nuclei on thin line patterns are exposed to maximum tension from actin stress fibres. Lateral nuclear compression on elongated patterns increases tension in the actin stress fibres which regulates the nuclear shape. As the actin stress fibres are connected to nucleus via LINC complex nesprins, FRET tension sensors report the tension between actin and nesprin. Thus, an increase in the tension of actin stress fibres will increase the tension at the LINC complex reported by low FRET intensities. Hence, it is possible that MSC nuclei on thinner patterns experience maximum tension from the actin stress fibers locally across the complete nuclear membrane, whereas MSC nuclei on thicker patterns experience varied levels of tension from actin stress fibres across the nuclear membrane.

Since increase in both tension and curvature increased YAP nuclear localization, it remains unclear which of these variables directly control YAP localization in MSCs. To differentiate between the two variables, osmotic pressures were used to compress and swell the nucleus and measure nuclear membrane tension, curvature, and YAP localization in MSCs confined on fibronectin coated glass coverslips. Nuclear membrane tension increased with both anisotropic nuclear compression and isotropic swelling, however, YAP Ratios increased only under anisotropic nuclear compression. This is because nuclear curvature only substantially increases under anisotropic compression and hence, it was concluded that the nuclear curvature is the most direct regulator of YAP nuclear localization in MSCs.

### Nuclear Curvature Determines Mesenchymal Stem Cell Fate

At this point only the role of nuclear curvature in regulating YAP nuclear localization in MSCs was observed, but its effect on cellular biochemistry and differentiation is still undetermined. It was therefore decided to examine the impact of nuclear curvature on MSC differentiation by confining MSCs on fibronectin lines of decreasing pattern widths to induced lateral nuclear compression and achieve variable degrees of nuclear curvature. MSCs were cultured in mixed induction medium that is supportive of both osteogenic and adipogenic fates. After two weeks in culture, MSCs were stained with Hoechst for nuclei, alkaline phosphatase (ALP) for osteogenic differentiation, and lipid molecules (Lpdx) for adipogenic differentiation.

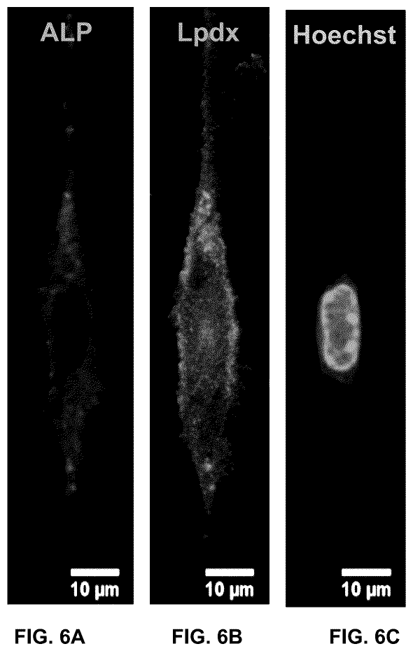

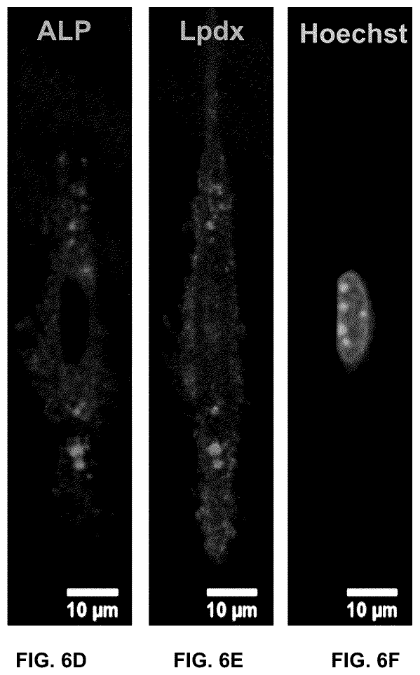

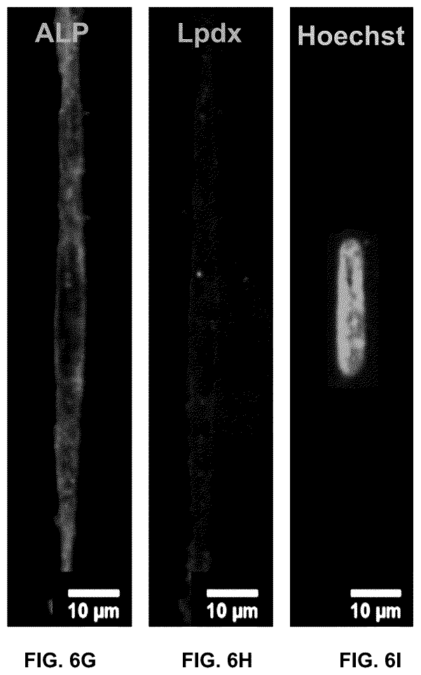

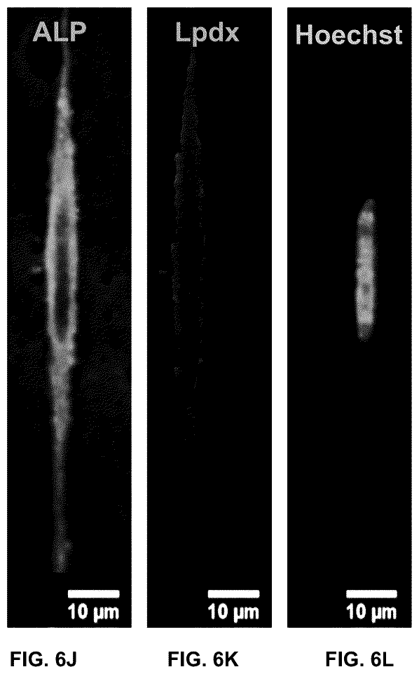

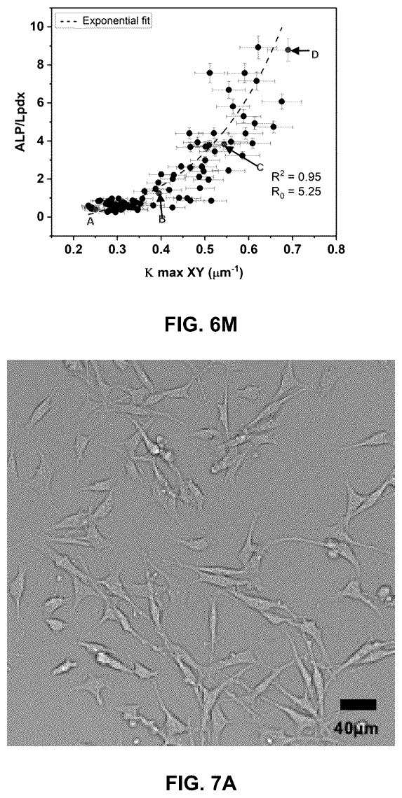

In MSCs on wide patterns with lower nuclear curvature, low ALP intensity and high Lpdx intensity was observed, signifying adipogenic differentiation (FIGS. 6A-6C); in stark contrast, on thin line patterns with high nuclear curvature high ALP intensity and low Lpdx intensity were observed exhibiting osteogenic differentiation. The transition between these two lineages appears as a continuous function of max curvature (FIGS. 6J-6L). The differentiation was quantified by calculating the total fluorescence intensity ratio of ALP and Lpdx for single MSCs and by comparing it with their maximum nuclear curvatures; with increasing Kmax XY an increase in ALP/Lpdx ratio was observed (FIG. 6M), thus indicating higher nuclear curvature favors osteogenic differentiation of MSCs (FIGS. 6G-6L), whereas lower nuclear curvature favors adipogenic differentiation of MSCs (FIGS. 6A-6F).

### DISCUSSION

MSC-based therapies have the potential to address some of the most conventionally untreatable diseases, however their application is predicated on the controlled differentiation of stem cells into their target cell type. In the present study YAP was identified as a key differentiation regulator. The biophysical aspects that regulate its localization and activity in MSCs have been demonstrated herein. Specifically, nuclear membrane curvature controls YAP nuclear localization in MSCs. Physiologically, this bending of the nuclear membrane was regulated by contractility induced tension at the nuclear membrane, which scales with nuclear bending and YAP nuclear localization. It was demonstrated that nuclear membrane curvature-mediated YAP localization is regulated by both active and passive nuclear import pathways by pharmacological modification of nuclear transport. Finally, it was demonstrated that MSC differentiation fate is determined by nuclear curvature, with low curvature inducing adipocyte differentiation and high curvature directing osteocyte lineage.

In previous studies it was reported that the force applied directly to the nucleus, increases nuclear flattening, stretches the nuclear pores and triggers YAP nuclear localization in epithelial cells. In the present study YAP nuclear localization was predominantly caused by nuclear curvature. This is when under hyperosmotic pressure nuclei in adherent cells are anisotropically compressed, with most deformation occurring in the Z direction (FIG. 3T). This further flattens the nucleus and increases nuclear curvature. Nevertheless, it is unclear how this nuclear curvature affects the nuclear pores or distribution of nuclear pores across the nuclear membrane and needs to be studied further.

Apart from the passive nuclear import via nuclear pores, a significant contribution from active nuclear import mechanisms was observed in nuclear curvature dependent localization of YAP (FIGS. 17A-17C). The active mechanisms rely on importin proteins that transport cargo proteins inside the nucleus in a RAN-GTP dependent manner. Importin 7 is an import protein for YAP, the localization of which is regulated by actomyosin contractility, where importin 7 localizes inside the nucleus with increasing contractility. Since, a similar nuclear localization of YAP was observed with contractility dependent increase in nuclear curvature (FIGS. 2A-2F), it is possible that importin 7 is also regulated by nuclear curvature and the role of nuclear deformation in regulation of importin 7 and YAP. Nuclear compression can also increase intracellular levels of calcium ions which can trigger localization of nucleoplasmic phospholipase A2 (cPLA2) to the nuclear membrane, which increases contractility and can affect both importin 7 and YAP activity.

In the present study it was also observed that with increasing nuclear curvature there is a nonlinear increase in YAP ratios in cells confined on thin line micropatterns (FIG. 4N), which was ascribed to lateral compression of nuclei. Generally cells confined on 1.5 μm patterned lines migrate in one-dimensional fashion, quantitatively mimicking morphology and migration in three-dimensional fibrillar microenvironment. This suggests that the 1D patterned lines here may faithfully reproduce some of the mechanotransduction experienced in fibrillar 3D environments.

Besides the proposed mechanism there may be additional aspects that regulate YAP nuclear localization with increasing nuclear curvature. One important factor that can regulate nuclear compression is the nuclear stiffness. Nuclear stiffness is in part determined by the expression and distribution of Lamin A/C, a part of nuclear lamins which are type V intermediate filaments localized under the nuclear membrane. Lamin A/C expression levels can affect both nuclear deformation and YAP nuclear localization. Fibroblast cells with overexpressed Lamin A/C and stiffer nuclei exhibit lower nuclear compression and YAP nuclear localization, whereas cells with silenced Lamin A/C and softer nuclei exhibit higher nuclear compression and YAP nuclear localization. Thus, Lamin A/C expression levels can directly influence nuclear curvature and nuclear membrane tension, affecting YAP nuclear localization. This makes it crucial to study the interplay between nuclear stiffness, curvature, membrane tension and YAP localization in MSCs.

Apart from the nuclear stiffness, nuclear lamins also determine the distribution of nuclear pore complexes on the nuclear membrane. Both A-type and B-type Lamins assemble into separate filament networks to form a composite structure on the nuclear membrane, providing attachment sites for nuclear pore complex proteins. In the present study passive nuclear import of YAP was shown via nuclear pore complexes with increasing nuclear curvature. In the present study it was also shown that YAP localizes inside the nucleus only under nuclear compression and not nuclear swelling. It is believed this is because of the anisotropic nuclear deformation and increase in nuclear curvature that happens only under nuclear compression and not swelling.

In conclusion, the present study correlates nuclear membrane bending with YAP nuclear localization and differentiation in MSCs. Additionally, the present demonstrated mechanism is consistent with YAP localization mechanisms via matrix mechanics, contractility, cytoskeletal strain transfers to nucleus, force induced nuclear flattening and stretch induced nuclear pore import in various cell types. Further the proposed mechanism is also consistent with mesenchymal stem cell differentiation mediated with cell compression, contractility and nuclear deformation. Hence, it was demonstrated that nuclear curvature is a strong determinant of YAP nuclear localization and differentiation in MSCs. Therefore nuclear curvature can be leveraged as a biophysical standard parameter to direct MSCs differentiation for stem cell therapies.

What is claimed is:

1. A method of selectively differentiating mesenchymal stem cells into a first predetermined cell lineage associated with the nuclear localization of Yes-associated protein (YAP) or into a second predetermined cell lineage associated with the cytoplasmic localization of YAP, the method comprising:
controlling the curvature of the nucleus to have a maximum nuclear curvature (Kmax) of at least 0.5 μm⁻¹ to select for the first predetermined cell lineage, or a Kmax that does not exceed 0.5 μm⁻¹ to select for the second predetermined cell lineage; and
incubating the mesenchymal stem cells having a controlled nuclear curvature in a media suitable for the differentiation mesenchymal stem cells obtain the first predetermined cell lineage or the second predetermined cell lineage.

2. The method of claim 1, wherein the media comprises differentiation additives.

3. The method of claim 1, wherein the first predetermined cell lineage is an osteocyte lineage.

4. The method of claim 1, wherein the second predetermined cell lineage is an adipocyte lineage.

5. The method of claim 1, wherein the second predetermined cell lineage is a neuronal lineage.

6. The method of claim 1, wherein the Kmax to select for the first predetermined cell lineage is at least 0.7 μm⁻¹.

7. The method of claim 1, wherein the controlling of the curvature comprises physically confining the mesenchymal stem cells.

8. The method of claim 7, wherein the confining comprises confining the mesenchymal stem cells on micro-patterns.

9. The method of claim 8, wherein the micro-patterns control cell adhesion promoting the cells ability to compress the nucleus and increasing YAP activity to select for the first predetermined cell lineage.

10. The method of claim 8, wherein the micro-patterns compress the nucleus of the mesenchymal stem cells.

11. The method of claim 8, wherein the micro-patterns control cell adhesion and restrict the cells ability to compress the nucleus and reducing YAP activity to select for the second predetermined cell lineage.

12. The method of claim 7, wherein the confining comprises confining the mesenchymal stem cells inside microchannels.

13. The method of claim 1, wherein the controlling of the curvature comprises applying a pressure on the nucleus of the mesenchymal stem cells.

14. The method of claim 13, wherein the pressure is applied vertically.

15. The method of claim 13, wherein the pressure is osmotic pressure.

16. The method of claim 15, wherein the osmotic pressure is applied using polyethylene glycol (PEG).

17. The method of claim 13, wherein the pressure is applied with a lateral nuclear compression using fibers.

18. The method of claim 17, wherein the fibers are electrospun, polymerized or extruded.

19. The method of claim 1, wherein the volume of the nucleus is controlled to be 50% less than the physiological volume of the nucleus to select for the first predetermined cell lineage.

20. The method of claim 2, wherein the differentiation additives are selected from β-glycerol phosphate, ascorbic acid, and dexamethasone.
